(19) United States
(12) Patent Application Publication
(10) Pub. No.: US 2016/0279110 A1
(43) Pub. Date: Sep. 29, 2016

(54) COMPOSITIONS AND METHODS FOR TREATING CANCER

(71) Applicants: Lynne E. Maquat, Rochester, NY (US); Maximilian W. Popp, Rochester, NY (US)

(72) Inventors: Lynne E. Maquat, Rochester, NY (US); Maximilian W. Popp, Rochester, NY (US)

(73) Assignee: UNIVERSITY OF ROCHESTER, Rochester, NY (US)

(21) Appl. No.: 15/082,750

(22) Filed: Mar. 28, 2016

Related U.S. Application Data
(60) Provisional application No. 62/138,783, filed on Mar. 26, 2015.

(51) Int. Cl. A61K 31/4375; A61K 31/704

(52) U.S. Cl. CPC A61K 31/4375; A61K 31/704

(57) ABSTRACT

Provided herein are compositions and methods for treating cancer.

This application claims the benefit of U.S. Provisional Application No. 62/138,783, filed Mar. 26, 2015, which is incorporated herein by reference in its entirety.

### STATEMENT REGARDING FEDERALLY FUNDED RESEARCH

This invention was made with Government Support under GM059614 awarded by the National Institutes of Health. The Government has certain rights in the invention.

### BACKGROUND

Cancer is a group of diseases characterized by abnormal cell growth with the potential to invade or spread to other parts of the body. In 2012, an estimated 14.1 million new cases of cancer and about 8.2 million cancer-related deaths occurred worldwide. Therefore, methods and compositions for treating cancer are necessary.

### SUMMARY

Provided herein are compositions and methods for treating cancer. The methods comprise administering to the subject with cancer a therapeutically effective amount of an agent that inhibits nonsense-mediated RNA decay (NMD) and a therapeutically effective amount of a chemotherapeutic agent.

Also provided are compositions and methods for increasing apoptosis in a cancer cell. The methods comprise contacting the cell with effective amount of an agent that inhibits nonsense-mediated RNA decay (NMD) and an effective amount of a chemotherapeutic agent.

Further provided are compositions and methods for increasing sensitivity of a cancer cell to a chemotherapeutic agent. The methods comprise contacting the cell with an effective amount of an agent that inhibits nonsense-mediated RNA decay (NMD) and an effective amount of a chemotherapeutic agent.

### DESCRIPTION OF THE DRAWINGS

FIGS. 1a-d show that NMD1 is inhibited during doxorubicin treatment. FIG. 1a shows mRNA decay assays in MCF7 cells. MCF7 cells either were (top line) or were not (bottom line) pretreated with 5 μM doxorubicin for 1 h before addition of 3 μg ml⁻¹ actinomycin D to halt transcription. Cells were collected at the indicated times after actinomycin D addition. Levels of the indicated NMD-targeted mRNAs were assessed by RT-qPCR, normalized to 18S rRNA levels and displayed as a percentage of the levels at t=0 h. Error bars=S.E.M, n==4 independent biological quadruplicates. FIG. 1b shows human β-Gl mRNA half-life studies in HeLa Tet-off cells. HeLa Tet-off cells were transfected with plasmids encoding human β-Gl Norm mRNA and MUP mRNA or β-Gl Ter mRNA and MUP mRNA. β-Gl Norm and β-Gl 39 Ter mRNA transcription occurs under the agency of the non-stress-responsive Tet-off promoter. Cells were either pretreated with nothing (top), 50 μM doxorubicin for 1 h (middle) or 50 μg ml⁻¹ puromycin for 3 h (bottom) before transcriptional shut-off with 2 μg ml⁻¹ doxycycline. Cell aliquots were removed at the indicated “chase” time points, and RT-qPCR was used to assess the remaining levels of β-Gl Norm and β-Gl Ter mRNAs, each after normalization to MUP mRNA. FIG. 1c shows Western blots of lysates of MCF7 cells from (FIG. 9a) (blots derive from and are representative of the three biological replicates in FIG. 9a that had been exposed to doxorubicin (5 μM) for the indicated times. GAPDH levels serve as loading controls. Threefold serial dilutions (wedge) reveal the dynamic range of analysis α, anti; 1-416, UPF1 amino acids; CP, cleavage product. FIG. 1d is as in c, but cells were exposed to a 10-fold higher concentration of doxorubicin and analysed at earlier time points. Data are representative of two biological replicates.

FIGS. 2a-c show that UPF1 CP production is an early and conserved event. FIG. 2a shows Western blots of lysates of HeLa cells (Homo sapiens) transfected with 100 nM of either control (Ctrl) siRNA or UPF1 siRNA and, 48 h later, exposed to CHX (300 μg ml⁻¹) for 3 h. FIG. 2b shows Western blots of lysates of HeLa cells (Homo sapiens) exposed to CHX (100 or 300 μg ml⁻¹) for either 3 or 5 h, or to ETP (44 μM) for 6 h, incubated in fresh medium and withdrawn from ETP at the indicated times FIG. 2c is essentially as in b, except C2C12 myoblasts (Mus musculus) were analysed. CHX concentrations were 100 or 300 μg ml⁻¹, and a 5 h pulse of ETP was used at 100 μM. At least two apoptotic inducers were used for each cell line, and at least two cell lines were tested with each apoptotic inducer.

FIGS. 3a-d show that UPF1 CP is produced by hydrolysis at aspartic acid 37 FIG. 3a shows Western blots of lysates of HEK293T cells preincubated with the indicated peptide-based fluoromethylketone caspase inhibitor for 4 h, before addition of CHX (300 μg ml⁻¹) for the indicated times. FIG. 3b is a diagram of N-terminally FLAG-tagged human UPF1, showing its reactivity with anti(α)-UPF1(1-416). CH, cysteine-t histidine-rich region. FIG. 3c shows Western blots of lysates of HeLa cells stably expressing an N-terminally FLAG-tagged UPF1 and incubated with CHX for the indicated times. FIG. 3d shows Western blots of lysates of HeLa cells retrovirally transduced at a multiplicity of infection <0.1 with either MYC-UPF1-FLAG WT or MYC-UPF1-FLAG D37N (and thus G418 resistant) and exposed to CHX (300 μg ml⁻¹) or doxorubicin (50 iμM) in the absence of G418. Blots are representative of two independent experiments.

FIGS. 4a-d show that UPF1 CP is not functional in NMD. FIG. 4a is a schematic of TCRβ reporter plasmids. Bidirectional (two minimal cytomegalovirus) promoters (horizontal arrows) drive expression of H-A-Cerulean protein (whose transcript is not an NMD target) and 3 FLAG-mCherry protein. The TCRβ JC intron either does (+) or does not (Δ) reside >55 nt downstream of the 3×-FLAG-mCherry termination codon (Ter), so that +JC intron transcripts are NMD targets, whereas ΔJC intron transcripts are not pA, polyadenylation signal. FIG. 4b shows Western blots of lysates of HEK293T cells transfected with either control (Ctrl) siRNA or UPF1 siRNA (100 nM) and, 24 h later, with (i) the MUP reference plasmid, (ii) either the β-Gl Norm reporter plasmid plus the TCRβ reporter plasmid lacking the JC intron (ΔJC intron) or the β-Gl Ter reporter plasmid plus the TCRβ reporter plasmid containing the JC intron (−JC intron) and (iii) one of the following UPF1 variants: MYC-UPF1-FLAG WT (lanes 7, 8, 11, 12, 19, 20); MYC-UPF1-FLAG D37N (lanes 13, 14); Δ37-UPF1-FLAG (lanes 21, 22); MYC-UPF1-FLAG TEV (lanes 23.24): MYC-UPF1 dNT (lanes 25, 26); or MYC-UPF1 R843C (lanes 27, 28). The level of each UPF1 variant was normalized to the level of GAPDH (triangles), while the level of 3×-FLAG-mCherry was normalized to the level of HA-Cerulean (circles). Blots derive from (and are representative of) the triplicate samples analysed in c. FIG. 4c shows RT-qPCR of RNA from samples analysed in b where β-Gl mRNA levels were normalized to the level of MUP mRNA, and the normalized level of β-Gl Norm mRNA in the presence of each UPF1 variant is defined as 100% FIG. 4d shows RT-qPCR of RNA from samples analysed in b where 3× FLAG-mCherry-TCRβ mRNA levels were normalized to the level of HA-Cerulean mRNA and the normalized level of 3. FLAG-mCherry-TCRβ ΔJC intron mRNA in the presence of each UPF1 variant is defined as 100%. Error bars=S.E.M., *=P<0.05 relative to the UPF11 siRNA+MYC-UPF1-FLAG WT sample using the Student's t-test. n=3 independent biological replicates.

FIGS. 5a-5c show that UPF1 CP dominantly interferes with NMD at substoichiometric levels FIG. 5a shows Western blots of lysates of HeLa cells transiently expressing increasing amounts of MYC-UPF1-FLAG WT or Δ37-UPF1-FLAG together with either the ‘Norm’ plasmid set (producing β-Gl Norm mRNA, 3 FLAG-mCherry-TCR(ΔJC intron mRNA, and MUP mRNA) or the ‘Ter’ plasmid set (producing β-Gl Ter mRNA, 3×-FLAG-mCherry-TCRβ+JC intron mRNA and MUP mRNA). Control experiments used empty vector (θ) in place of a UPF1 variant Anti(α)-FLAG immunoblot allows unambiguous comparison of MYC-UPF1-FLAG WT and Δ37-UPF1-FLAG levels: Δ37-UPF1-FLAG exhibits about one-third the immunoreactivity of full-length UPF1 with anti-UPF1(1-416) antiserum. Blots derive from (and are representative of) the triplicate samples analysed in b,c. FIG. 5b shows RT-qPCR, where the level of β-Gl Norm or Ter mRNA was first normalized to the level of MUP mRNA, and subsequently normalized to the empty-vector (θ) control (defined as 100%). The arrow denotes the level of Δ37-UPF1-FLAG (based on a) that is comparable to the physiological level of UPF1 CP. FIG. 5c shows RT-qPCR, where the level of 3: FLAG-mCherry-TCRβ ΔJC intron mRNA or 3×-FLAG-mCherry-TCRβ+JC intron mRNA was first normalized to the level of MUP mRNA, and subsequently normalized to the empty-vector (θ) control (defined as 100%). The arrow denotes the level of Δ37-UPF1-FLAG (based on a) that is comparable to the physiological level of UPF1 CP. Error bars=S.E.M., *=P<0.05 relative to no UPF1 variant (empty vector) sample using the Student's t-test. n=3 independent biological replicates.

FIG. 6 shows characterization of the UPF1 CP mRNP. Western blots of lysates of HEK293T cells transfected with either control (Ctrl) siRNA or UPF1 siRNA (100 nM) and 24 h later with plasmid encoding MYC-UPF1-FLAG WT or Δ37-UPF1-FLAG either before (Input) or after anti(α)-FLAG IP, the latter in the presence of bovine serum albumin (−) or RNase ONE (+). Blots are representative of two independent experiments.

FIGS. 7a-e show generation of UPF1 CP in the absence of doxorubicin upregulates genes that promote the apoptotic response to doxorubicin. FIG. 7a shows a Western blot of lysates of HeLa cells retrovirally transduced with empty vector (θ) or the MYC-UPF1-FLAG TEV construct bearing a substitution of the TEV protease cleavage site (ENLYFQS) at D37, and subsequently transiently transfected with empty vector (θ) or a vector expressing both halves of TEV protease (each half is MYC-epitope tagged). Both full-length MYC-UPF1-FLAG TEV and the resultant TEV-generated CP retain the C-terminal FLAG. Blots derive from (and are representative of) duplicate samples analysed by RNA-seq. For FIG. 7b. HeLa cells were transfected as in FIG. 5a, but without NMD reporter sets. Blots derive from (and are representative of) the triplicate samples analysed in c. FIG. 7c shows RT-qPCR of mRNA from cells in b where the levels of the indicated transcripts in each transfectant were first normalized to the level of GAPDH mRNA, and the ratio of normalized transcript levels in the Δ37-UPF1-FLAG transfectant to the normalized transcript levels in cells expressing an equivalent amount of MYC-UPF1-FLAG WT are displayed. The normalized transcript levels in the MYC-UPF1-FLAG WT sample for each amount of transfected plasmid is set at 100%. FIG. 7d is essentially as in b but using MCF7 cells. Blots derive from (and are representative of) the triplicate samples analysed in e. FIG. 7e is essentially as in c, but using mRNA from MCF7 cells in d. Error bars=S.E.M., *=P<0.05 for Δ37-UPF1-FLAG sample relative to MYC-UPF1-FLAG WT sample using the Student's t-test. n=3 independent biological replicates.

FIGS. 8a-e show that inhibiting UPF1 CP generation protects cells from doxorubicin challenge and inhibiting NMD promotes doxorubicin-induced cell death. FIG. 8a shows Western blots of HeLa cells stably expressing empty vector (θ) or equivalent amounts of MYC-UPF1-FLAG WT or MYC-UPF1-FLAG D37N. Blots derive from (and are representative of) four biological replicate samples used in b. In FIG. 8b, cell lines from a were plated in 96-well opaque tissue-culture dishes (5,000 cells per well) and exposed to the indicated concentrations of doxorubicin for 16 h. Viable cells were quantitated using a Cell Titer Glo assay. Data are normalized to untreated cells for each cell line. Errors bar=S.E.M., *=P<0.05 relative to MYC-UPF1-FLAG WT cell line samples using the Student's t-test. n=4 biological replicates. FIG. 5c shows the structure of NMDI-1. (d) HeLa cells were plated as in b and exposed to one of three treatments 24 h later—the indicated doxonibicin concentration is provided for 16 h either alone (left histogram) or in the presence of 10 μM NMDI-1 (middle histogram). Alternatively, cells were preincubated with 10 M NMDI-1 for 8 h, washed three times and incubated with the indicated concentrations of doxorubicin for 16 h in the absence of NMDI-1. Viable cells were quantitated as in b. Error bars=S.E.M, *=P<0.05 relative to doxorubicin alone treatment using the Student's t-test. n=4 biological replicates. Figure Se shows a model implicating NMD modulation in establishing different cellular states (compare box to the left and box to the right) by sculpting the mRNA milieu. Transcription produces mRNAs that are or are not NMDI) targets (that is, are unregulated) or are indirect NMD targets. Normally, NMD is active, eliminating direct and indirect NMD targets from the mRNA milieu (left box). However, NMD activity can be modulated by various perturbations. Shown here is that production of UPF1 CP(s) at substoichiometric levels downregulates NMDI) activity, causing direct NMD targets to enter the mRNA milieu, which secondarily causes upregulation of indirect NMD targets (right box). Together these changes sculpt the pool of m RNAs to one that is competent to rapidly respond to the stimulus that elicited the inhibition of NMD. Here we have shown that the stimulus (doxorubicin) attenuates NMD, facilitating an appropriate response (cell death).

FIGS. 9a and 9b show that the mRNA/pre-mRNA ratio, as assessed using RT-quantitative PCR (qPCR), increases for several transcripts (none of which is known to be stress-regulated), in response to doxorubicin. In FIG. 9a, MCF7 cells were treated with 5 M doxorubicin for the hours (h) indicated. mRNA-to-pre-mRNA ratios for the specified transcripts were measured using RT-qPCR and normalized to the ratio at 0 h, which is defined as 100%. The level of each pre-mRNA was also normalized to the level of GAPDH mRNA, and the normalized level at 0 h is defined as 100%. There are separate scales for the graphs of mRNA/pre-mRNA ratios (left) and the graphs of pre-mRNA/GAPDH mRNA ratios (right). Separately, MCF7 cells were transfected with plasmids encoding reporter β-Gl Norm mRNA or β-Gl Ter mRNA together with a plasmid encoding the reference MUP mRNA and subsequently exposed to doxorubicin (5 μM) for the times indicated. Reporter transcript levels were normalized to the level of MUP mRNA, and the normalized level of β-Gl Ter mRNA was presented as a percentage of the normalized level of β-Gl Norm mRNA, which is defined as 100% at 0 h. Error bars=S.E.M., asterisk=p<0.05 relative to the 0 h time point using the Student's t-test. #=pre-mRNA levels below qPCR detection threshold. n>3 independent biological replicates. FIG. 9b shows that CDKN1A, GADD45α, and GADD45βmRNAs are NMD targets in both HeLa and MCF7 cells. HeLa and MCF7 cells were transfected with either control (Ctrl) siRNA or UPF1 siRNA, the latter to deplete endogenous UPF1 protein to less than 5% of the normal level (top western blots). mRNA/pre-mRNA ratios were then assessed using RT-qPCR as in FIG. 1a (bottom). Western blots derive from and are representative of 3 independent biological replicates used for the corresponding RT-qPCR analyses. Error bars=S.E.M., asterisk=p<0.05 relative to control siRNA using the Student's t-test. n=3 independent biological replicates.

FIGS. 10a-h show that UPF1 CP production is an early and conserved apoptotic event. FIG. 10a shows Western blotting of lysates of human HEK293T cells exposed to 300 μg/mL cycloheximide (CHX) for the indicated times. α, anti; h, hour(s). FIG. 10b is essentially as in a, but using lysates of HEK293T cells exposed to 1 μM staurosporine. FIG. 10c is essentially as in a, but using lysates of human Daudi B lymphoblasts exposed to 40 ng/mL TNF-α or 2 μg/mL doxorubicin. FIG. 10d is essentially as in a, but using lysates of human Jurkat T cells exposed to 1 μM staurosporine. FIG. 10e is essentially as in a, but using lysates of canine MDCK cells exposed to 2 μM staurosporine. FIG. 10f is essentially as in a, but using lysates of bovine MDBK cells exposed to 2 μM staurosporine. FIG. 10g is essentially as in a, but using lysates of Chinese Hamster Ovary (CHO) cells exposed to 2 μM staurosporine. FIG. 10h is essentially as in a, but using lysates of African green monkey COS-7 cells exposed to 2 μM staurosporine or 2 μg/mL doxorubicin. At least two apoptotic inducers were used for each cell line, and at least two cell lines were tested with each apoptotic inducer.

FIGS. 11a-d show that UPF1 is cleaved at position D37. FIG. 11a shows a Western blot of lysates of human HeLa cells transiently transfected with a plasmid encoding MYC-UPF-FLAG protein harboring no mutation (WT) or an asparagine (N) substitution at aspartic acid 27 (D27N), 37 (D37N), or 75 (D75N) and, 48 h later, exposed to 300 μg/mL CHX to induce UPF1 CP generation and apoptosis. UPF1 CP may result from cleavage of endogenous UPF1 or the transfected construct, but the use of anti-FLAG allows unambiguous assignment of UPF1-FLAG CP as derived from MYC-UPF1-FLAG. α, anti; h, hour(s). FIG. 11b shows a Western blot of lysates of MCF7 cells stably expressing MYC-UPF1-FLAG WT exposed to 5 μM doxorubicin for 16 h to generate the UPF1-FLAG CP. To serve as molecular weight standards for UPF1 CP, lysates of MCF7 cells transiently expressing a UPF1-FLAG protein encompassing the residues specified were also analyzed. Here again, use of anti-FLAG shows unambiguous molecular weight assessment of the doxorubicin-generated UPF1-FLAG CP relative to the transfected constructs. FIG. 11c shows ClustalXgenerated multiple sequence alignments of Saccharomyces cerevisiae (baker's yeast), Schizosaccharomyces pombe (fission yeast), Bos taurus (cow), Xenopus tropicalis (Western clawed frog), Homo sapiens (human), Mus musculus (mouse), Gallus gallus (chicken), Drosophila melanogaster (fruit fly), and Caenorhabditis elegans (roundworm) UPF1 proteins. The experimentally demonstrated S10 and T28 phospho-acceptor residues (numbers correspond to the human sequence) are shown. The D37 cleavage site encompassing P4-P1 residues is boxed. FIG. 11d shows a Western blot. Immunoblots are representative of at least 2 independent experiments.

FIGS. 12a and 12b show that, at sub-stoichiometric levels, UPF1 CP dominantly interferes with NMD in HEK293T cells. FIG. 12a is a Western blot of lysates of HEK293T cells in which endogenous UPF1 was challenged by transient transfection with (i) increasing amounts of plasmid encoding MYC-UPF1-FLAG WT (lanes 5-8, 17-20) or Δ37-UPF1-FLAG (lanes 9-12, 21-24), or empty vector (θ), (ii) the reference MUP plasmid and (iii), to assay for NMD, either β-Gl Norm (right) or β-Gl Ter (left) plasmid. Use of anti(α)-FLAG allows unambiguous comparison of MYCUPF1-FLAG WT and Δ37-UPF1-FLAG levels, since Δ37-UPF1-FLAG exhibits about one-third the immunoreactivity of full-length UPF1 with anti-UPF1(1-416). Blots derive from (and are representative of) the triplicate samples analyzed in b. FIG. 12b shows the levels of β-Gl Ter mRNA, quantitated as in FIG. 4b, where the normalized level in the absence of an exogenous UPF1 variant, i.e., empty vector, is defined as 100%. Error bars=S.E.M., asterisk=p<0.05 relative to the UPF1 siRNA+MYC-UPF1-FLAG WT sample using the Student's t-test. n=3 independent biological replicates.

FIG. 13 shows that MYC-UPF1-FLAG WT, Δ37-UPF1-FLAG, and MYC-UPF1-FLAG D37N proteins are enriched on PTC-containing transcripts relative to the PTC-free counterpart. HEK293T cells were transfected with a plasmid encoding the indicated UPF1 variant, as well as a plasmid encoding either β-Gl Norm and MUP mRNAs (Norm set) or β-Gl 39 Ter and MUP mRNAs (Ter set). Cells were harvested 48 h later, lysed, and proteins and associated RNAs were retrieved via anti-FLAG immunoprecipitation (IP). Immunoblots (top) were performed to indicate that the expression of each variant before (−) IP was comparable to endogenous UPF1 and that anti-FLAG immunoprecipitation retrieved equivalent amounts of each variant. β-Gl and MUP mRNA levels were assessed for each sample using RT-qPCR and β-Gl mRNA levels were normalized to the levels of MUP mRNA. Each normalized level of β-Gl mRNA after (+IP) IP was divided by the normalized level of β-Gl mRNA before IP and displayed (bottom). Immunoblots derive from and are representative of n=3 independent biological replicates used for the corresponding RT-qPCR analyses.

FIGS. 14a and 14b show that, relative to MYC-UPF1-FLAG WT, Δ37-UPF1-FLAG expression stabilizes endogenous NMD targets. In FIG. 14a, HeLa cells were transfected with the indicated constructs and 48 h later treated with 100 μg/ml 5,6-dichloro-1-β-D-ribofuranosyl-1H-benzimidazole (DRB). Cells were then harvested at the time points indicated below during the “chase” period. Western blotting was performed to assess expression levels. Blots were derived from (and are representative of) three biological replicates analyzed in (b). In FIG. 14b, RT-qPCR was used to assess the levels of the indicated transcripts from cells in (a) after DRB treatment. Transcript levels were normalized to 18S rRNA. The time where levels reached 50% of the starting amount is indicated. Error bars=S.E.M. n=3 biological replicates.

FIGS. 15a-d show that UPF1 CP causes sensitivity to doxorubicin in both HeLa and MCF7 cells. FIG. 15a is a Western blot of lysates of HeLa cells transiently transfected with plasmid encoding empty vector (θ), MYC-UPF1-FLAG WT, or the Δ37-UPF1-FLAG CP and, after 16 h, plated into 60-mm dishes (329,000 cells/plate) and incubated for an additional 24 h. The use of anti(α)-FLAG allows unambiguous comparison of MYC-UPF1-FLAG WT and Δ37-UPF1-FLAG levels, since Δ37-UPF1-FLAG exhibits about one-third the immunoreactivity as full-length UPF1 using anti-UPF1(1-416). FIG. 15b is essentially as in a, and performed in parallel to a, but cells were plated into opaque 96-well plates (5,000 cells/well, which is the same density as in a, and exposed to the indicated concentration of doxorubicin for 16 h. Cells were then washed to remove doxorubicin, and viable cells were quantitated using the Cell-Titer Glo assay. To control for variations in plating efficiencies, the data are normalized to untreated cells for each transfected cell line. Error bars=S.E.M., asterisk=p<0.05 relative to MYC-UPF1-FLAG WT cells using the Student's t-test. n=4 biological replicates. FIG. 15c is a Western blot of lysates of MCF7 cells transiently transfected as in a. However, cells were plated into 60-mm dishes (658,000 cells/plate). FIG. 15d is essentially as in c, and performed in parallel to c, but cells were plated into opaque 96-well plates (10,000 cells/well, which is the same density as in c, and exposed to doxorubicin, assayed for viability, and controlled for variations in cell plating as in b.

FIGS. 16a-d show that NMD-1 is a potent inhibitor of NMD in HeLa cells but not MCF7 cells. FIG. 16a shows RT-qPCR of RNA from lysates of HeLa cells transfected with the Norm or Ter plasmid set described in FIG. 4a, but TCRβ ΔJC intron and TCRβ+JC intron reporter plasmids were substituted for GPx1 Norm and GPx1 Ter reporter plasmids, respectively. Norm encodes a PTCfree version of GPx1 mRNA, whereas Ter encodes mRNA harboring a PTC at codon 46. Cells were re-plated 16 h after transfection at equivalent densities and exposed 24 h later to the indicated concentration of NMDI-1 or, as a positive control for NMD inhibition, cycloheximide (CHX). The levels of β-Gl reporter mRNAs were normalized to the level of the MUP reference mRNA, and the normalized levels of β-Gl Ter mRNA are presented as a percentage of the normalized level of β-Gl Norm mRNA. FIG. 16b is essentially as in a, except GPx1Ter mRNA was analyzed. FIG. 16c is essentially as in b, except MCF7 cells were used. FIG. 16d is essentially as in b, except MCF7 cells were used. For all, errors bar=S.E.M., asterisk=p<0.05 relative to no treatment samples using the Student's t-test. n=3 biological replicates.

### DETAILED DESCRIPTION

An estimated one-third of inherited diseases are the result of premature termination codon (PTC) acquisition. Nonsense-mediated mRNA decay (NMD) is a conserved mRNA quality control pathway deployed by cells to eliminate mRNAs containing a PTC. As used throughout, the term nonsense-mediated mRNA decay (NMD) refers to a process that controls the quality of eukaryotic gene expression and also degrades physiologic mRNAs. This process limits the production of aberrant mRNAs containing a premature termination codon and also controls the levels of endogenous transcripts.

Because proteins produced by PTC-containing mRNAs may have deleterious consequences, selection and destruction of these mRNAs by NMD maintains cellular homeostasis. Less well understood, but equally important, is the role of NMD in maintaining and regulating the levels of endogenous, non-mutated transcripts. These transcripts are of heterogeneous structure and encode proteins of heterogeneous function, yet they have the unifying feature that disrupting NMD elicits their upregulation. How NMD-mediated changes in the levels of these transcripts are integrated into cellular physiology is generally unclear.

Destruction of NMD targets is the result of incompletely understood mRNP rearrangements. Target selection is strictly dependent on translation and the result of one of at least five classes of cis-residing transcript features: (i) an upstream open reading frame (uORF) in the 5′ UTR where the stop codon of the uORF is a PTC relative to the main ORF; (ii) a shift in the translational reading frame because of alternative pre-mRNA splicing that generates a PTC≧50-55 nts upstream of an exon-exon junction or occurs ≧50-55 nts downstream of the normal termination codon, in either case so that an exon-junction complex (EJC) of proteins deposited upstream of an exon-exon junction fails to be removed by translating ribosomes; (iii) abnormally long 3′ UTRs; (iv) a UGA codon within specialized selenoprotein mRNAs that encodes selenocysteine with less than 100% efficiency, resulting in PTC-triggered NMD; or (v) a natural stop codon ≧50-55 nts upstream of a splicing generated exon-exon junction. In a model of EJC-mediated NMD, the terminating ribosome nucleates a complex termed “SURF”, which is composed of the protein kinase SMG1, the key NMD factor UPF1, and eukaryotic release factors (eRFs)1 and 3, on the mRNA to be degraded. The EJC is decorated with NMD factors UPF3 or UPF3× and UPF2 that, upon UPF1 binding, promote the ATP-dependent RNA helicase activity of UPF1. The defining feature of an mRNA that is destined for destruction is the presence of phosphorylated UPF1 (p-UPF1). SMG1 phosphorylates human UPF1 in both its N- and C-terminal tails. p-UPF1 then recruits SMG6 RNA endonuclease, and/or SMG5-SMG7 or SMG5-PNRC2 complexes, the latter two of which further recruit RNA deadenylating and decapping activities that precede exonucleolytic activities.

Administration of small molecule anti-cancer drugs is a mainstay of cancer treatment. Among the drugs used are topoisomerase inhibitors which cause double-stranded DNA breaks. DNA damage activates the p53 tumor-suppressor pathway, an early consequence of which is the inhibition of cell division. In cases of severe DNA damage, regulated cell death or apoptosis ensues. As shown herein, NMD is integrated into the network of processes that define the apoptotic response. As disclosed herein, NMD is inhibited during apoptosis, in part by the proteolytic production of a dominant-interfering form of UPF1. Inhibiting UPF1 cleavage protects cells from the effects of a chemotherapeutic agent. Conversely, decreasing the efficiency of NMD using a small molecule inhibitor sensitizes cells to a chemotherapeutic agent. Therefore, NMD can be modulated in order to treat cancer in a subject.

Provided herein is a method of treating cancer in a subject comprising administering to the subject with cancer a therapeutically effective amount of an agent that inhibits nonsense-mediated RNA decay (NMD) and a therapeutically effective amount of a chemotherapeutic agent. The agent that inhibits NMD can be any inhibitor of NMD now known or identified in the future. Inhibition of NMD can occur by, for example and not to be limiting, by decreasing phosphorylation of UPF1, inhibiting SMG1 and/or inhibiting UPF1 binding to a PTC-containing mRNA. Inhibition of NMD can also be effected by inhibiting the interaction between UPF1 and SMG5. For example, a chemical, a drug, a peptide, a protein, an antibody, an antisense RNA, an siRNA, a morpholino, a locked nucleic acid (LNA), an miRNA or a small molecule can be used to inhibit NMD. For example, a small molecule such as NMDI-1, as shown below, can be used (See Gotham et al. “Synthesis and activity of a novel inhibitor of nonsense-mediated mRNA decay,” Org. Biomol. Chem. 14(5): 1559-1563 (2016)) to inhibit NMD.

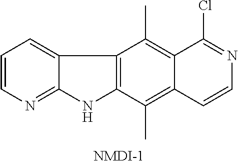 [SMILES: Cc1c2ccnc(Cl)c2c(C)c2c1[nH]c1ncccc12]

Other examples include, but are not limited to, pateamine A (See, Dang et al. “Inhibition of nonsense-mediated mRNA decay by the natural product pateamine A through eukaryotic initiation factor 4AIII,” J. Biol. Chem. 284(35): 23613-21 (2009)); and VG1, as shown below (See Gotham et al.).

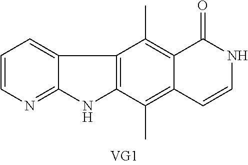 [SMILES: Cc1c2cc[nH]c(=O)c2c(C)c2c1[nH]c1ncccc12]

An NMD inhibitor having the formula set forth below, available as Product No. 530838 from Calbiochem (Temecula, Calif.), can also be used. Derivatives and salts of all of the agents provided herein can also be used in the methods provided herein. Any of the agents provided herein can also be modified to enhance cell permeability while maintaining the ability to inhibit NMD.

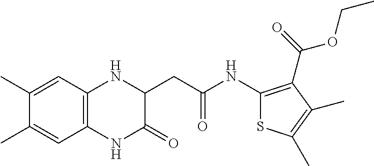 [SMILES: CCOC(=O)c1c(NC(=O)CC2Nc3cc(C)c(C)cc3NC2=O)sc(C)c1C]

In the methods provided herein, the agent that inhibits NMD can be administered to a subject prior to, simultaneously, or after administration of the chemotherapeutic agent. For example, the agent that inhibits NMD can be administered to a subject 30 minutes, one hour, two hours, three hours, four hours, five hours, six hours, seven hours, eight hours, nine hours, ten hours, eleven hours, twelve hours, thirteen hours, fourteen hours, fifteen hours, sixteen hours, seventeen hours, eighteen hours, nineteen hours, twenty hours, twenty-one hours, twenty-two hours, twenty-three hours, twenty-four hours, one day, two days, three days, four days, five days, six days, a week, two weeks, three weeks, four weeks or more prior to or after administration of the chemotherapeutic agent.

In any of the methods provided herein, the effective amount of one or more chemotherapeutic agents administered to a subject can be lower than the effective amount of the one or more chemotherapeutic agents when administered alone or in the absence of the agent that inhibits NMD. The effective amounts of both the agent that inhibits NMD and the chemotherapeutic agent can be lower as compared to the effective amount when either agent is administered alone or in the absence of the other agent to treat cancer.

By way of example, dosages of doxorubicin used alone in treating cancer of breast, ovary, prostate, stomach, thyroid; small cell cancer of lung, liver; squamous cell cancer of head and neck; multiple myeloma, Hodgkin's disease, lymphomas, ALL, AML can be calculated as 60-75 mg/m² IV every 21 days, 60 mg/m² IV every 14 days, 40-60 mg/m² IV every 21-28 days, or 20 mg/m² IV every week. Such dosages are modified when serum bilirubin levels are greater than 1.2 mg/dL. For example, a 50% dosage of doxorubicin is used when serum bilirubin levels are 1.2-3 mg/dL, and a 25% dosage is used when serum bilirubin levels are 3.1-5 mg/dL). When used in combination with NMD inhibitors, such dosages of doxorubicin can be reduced by 5, 10, 20, 30, 40, 50, 60 percent or more or the frequency of treatment can be reduced.

As used throughout, chemotherapeutic agents are compounds which can inhibit the growth of cancer cells or tumors. It is understood that one or more chemotherapeutic agents can be used in any of the methods set forth herein. For example, two or more chemotherapeutic agents, three or more chemotherapeutic agents, four or more chemotherapeutic agents, etc. can be used in the methods provided herein. Combinations of chemotherapeutic agents comprising doxorubicin are exemplary combinations that can be used in the methods provided herein. The chemotherapeutic agents that can be used include, but are not limited to, antineoplastic agents such as Acivicin; Aclarubicin; Acodazole Hydrochloride; AcrQnine; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflomithine Hydrochloride; Elsamitrucin; Enloplatin; Enpromate; Epipropidine; Epirubicin; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; 5-Fluorouracil; Flurocitabine; Fosquidone; Fostriecin Sodium; Gemcitabine; Gemcitabine Hydrochloride; Gold Au 198; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin C; Mitosper; Mitotane; Mitoxantrone; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safmgol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride.

Further examples of chemotherapeutic agents include 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; atrsacrine; anagrelide; anastrozole; andrographolide; angiogenesis inhibitors; antagonist D; antagonist G; antarelix; anthracyclines; anti-dorsalizing morphogenetic protein-1; antiandrogens, prostatic carcinoma; antiestrogens; antineoplastons; antisense oligonucleotides; aphidicolin glycinate; apoptosis gene modulators; apoptosis regulators; apurinic acid; ara-CDP-DL-PTBA; arginine deaminase; aromatase inhibitors; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docosanol; dolasetron; doxifluridine; droloxifene; dronabinol; duocannycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; fmasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hormone therapies; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; irinotecan; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; LHRH analogs; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance genie inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; progestational agents; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; rub oxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfmosine; superactive vasoactive intestinal peptide antagonist; suradista; suramin; swainsonine; synthetic glycosaminoglycans; tallimustine; tamoxifen; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene dichloride; topotecan; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer.

As used throughout, by subject is meant an individual. For example, the subject is a mammal, such as a primate, and, more specifically, a human. Non-human primates are subjects as well. The term subject includes domesticated animals, such as cats, dogs, etc., livestock (for example, cattle, horses, pigs, sheep, goats, etc.) and laboratory animals (for example, ferret, chinchilla, mouse, rabbit, rat, gerbil, guinea pig, etc.). Thus, veterinary uses and medical uses and formulations are contemplated herein. The term does not denote a particular age or sex. Thus, adult and newborn subjects, whether male or female, are intended to be covered. As used herein, patient or subject may be used interchangeably and can refer to a subject afflicted with a disease or disorder.

As used herein, cancer can be, but is not limited to, neoplasms, which include solid and non-solid tumors. A neoplasm can include, but is not limited to, pancreatic cancer, breast cancer, head and neck cancer, ovarian cancer, melanoma, bladder cancer, bone cancer, brain cancer (e.g., glioblastoma or neuroblastoma), lung cancer, prostate cancer, colon cancer, cervical cancer, esophageal cancer, endometrial cancer, central nervous system cancer, gastric cancer, colorectal cancer, thyroid cancer, renal cancer, oral cancer, Hodgkin lymphoma, skin cancer, adrenal cancer, liver cancer, and leukemia. Cancers also include cancers that affect the hematopoietic system, for example, B-cell cancers, such as multiple myeloma or lymphoma.

As used herein, the terms treatment, treat, treating or ameliorating refers to a method of reducing one or more effects of a disease or condition or one or more symptoms of the disease or condition, including a recurrence of the disease or condition. Thus in the disclosed method, treatment can refer to a 10%, 20%, 30%, 40%, 50%, 60%, 70%, 80%, 90%, or 100% reduction or amelioration in the severity of an established disease or condition or symptom of the disease or condition. For example, the method for treating cancer is considered to be a treatment if there is a 10% reduction in one or more symptoms of the disease in a subject as compared to control. Thus, the reduction can be a 10, 20, 30, 40, 50, 60, 70, 80, 90, 100%, or any percent reduction in between 10 and 100 as compared to for example, a subject that is treated with the chemotherapeutic agent in the absence of the agent that inhibits NMD or an untreated subject. It is understood that treatment does not necessarily refer to a cure or complete ablation of the disease, condition, or symptoms of the disease or condition.

Further provided are methods of increasing apoptosis in a cancer cell or population of cancer cells comprising contacting the cell or cells with effective amount of an agent that inhibits NMD and an effective amount of a chemotherapeutic agent. The treated cell or cells, by way of example, can be treated ex vivo for transplantation to the same subject or to a different subject from which the cells are derived. As used throughout, a cell can be in vitro, in vivo or ex vivo. Optionally the cells to be treated are present in a biological sample such as a bone marrow sample, a blood sample, or the like.

The cell can be contacted with the agent that inhibits NMD prior to, simultaneously with, or after administration of the chemotherapeutic agent. For example, a cell can be contacted with the agent that inhibits NMD 30 minutes, one hour, two hours, three hours, four hours, five hours, six hours, seven hours, eight hours, nine hours, ten hours, eleven hours, twelve hours, thirteen hours, fourteen hours, fifteen hours, sixteen hours, seventeen hours, eighteen hours, nineteen hours, twenty hours, twenty-one hours, twenty-two hours, twenty-three hours, twenty-four hours, one day, two days, three days, four days, five days, six days, a week, two weeks, three weeks, four weeks or more prior to contacting the cell with a chemotherapeutic agent.

As used throughout, apoptosis refers to a process that is characterized by biochemical events that lead to morphological changes in cells and cell death. These changes include among others, blebbing, cell shrinkage, nuclear fragmentation, chromatin condensation and chromosomal DNA fragmentation.

As used throughout an increase in apoptosis can be greater than about a 10%, 20%, 30%, 40%, 50%, 60%, 70% 10%, 20%, 30%, 40%, 50%, 60%, 70%, 80%, 90%, or more, as compared to a control or can constitute complete ablation of target cells. The increase in apoptosis can also be greater than about 2-fold, 5-fold, 10-fold, 25-fold, 50-fold, 75-fold, 100-fold, 125-fold, 150-fold, 175-fold, 200-fold, 225-fold, 250-fold, 300-fold or more a compared to a control.

Also provided are methods of increasing the sensitivity of a cancer cell(s) to a chemotherapeutic agent comprising contacting the cell(s) with effective amount of an agent that inhibits nonsense-mediated RNA decay (NMD) and an effective amount of a chemotherapeutic agent. By way of example, the cancer cell can be treated with the NMD inhibitor prior to treatment with the chemotherapeutic agent.

As used throughout an increase in sensitivity can be greater than about a 10%, 20%, 30%, 40%, 50%, 60&, 70% 10%, 20%, 30%, 40%, 50%, 60%, 70%, 80%, 90%, 100% 200%, 300%, 400% or more as compared to a cell that is contacted with a chemotherapeutic agent in the absence of an agent that inhibits NMD or a cell that is resistant to the chemotherapeutic agent. The increase can also be greater than about 2-fold, 5-fold, 10-fold, 25-fold, 50-fold, 75-fold, 100-fold, 125-fold, 150-fold, 175-fold, 200-fold, 225-fold, 250-fold, 300-fold or more a compared to a control.

According to the methods taught herein, the subject is administered an effective amount of the agent(s) or the one or more cells are contacted with an effective amount of the agent(s). The terms effective amount, effective dosage, and therapeutically effective amount are used interchangeably. The term effective amount is defined as any amount necessary to produce a desired physiologic response. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, anaphylactic reactions, and the like. Generally, the dosage will vary with the activity of the specific compound employed, the metabolic stability and length of action of that compound, the species, age, body weight, general health, sex and diet of the subject, the mode and time of administration, rate of excretion, drug combination, and severity of the particular condition and can be determined by one of skill in the art. The dosage can be adjusted by the individual physician in the event of any contraindications. With regard to in vitro or ex vivo treatment of cells, the desired effects include inducing or increasing apoptosis and/or promoting or increasing the sensitivity of the cell to chemotherapeutic agents.

Dosage regime can vary, and can be administered in one or more dose administrations daily, for one or several days. Guidance can be found in the literature for appropriate dosages for given classes of pharmaceutical products. Optionally, the agent(s) are administered continuously or intermittently.

It should be noted that the effective amount and the dosage for the NMD inhibitor and the chemotherapeutic agent may be less when the agents are used together as compared to when the agents are used separately.

Also provided herein are compositions comprising one or more NMD inhibitors and one or more chemotherapeutic agents. The compositions can be in a pharmaceutically acceptable carrier. The term carrier means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

As used herein, the term carrier encompasses any excipient, diluent, filler, salt, buffer, stabilizer, solubilizer, lipid, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington: The Science and Practice of Pharmacy 22d edition Loyd V. Allen et al., editors, Pharmaceutical Press (2012). Examples of physiologically acceptable carriers include buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN® (ICI, Inc.; Bridgewater, N.J.), polyethylene glycol (PEG), and PLURONICS™ (BASF; Florham Park, N.J.).

Modes of administration of the agent(s) or composition(s) are discussed below. Any of the compositions described herein can be delivered by any of a variety of routes including: by injection (e.g., subcutaneous, intramuscular, intravenous, intra-arterial, intraperitoneal), by continuous intravenous infusion, cutaneously, dermally, transdermally, orally (e.g., tablet, pill, liquid medicine, edible film strip), by implanted osmotic pumps, by suppository, or by aerosol spray. Routes of administration include, but are not limited to, topical, intradermal, intrathecal, intralesional, intratumoral, intrabladder, intravaginal, intra-ocular, intrarectal, intravesicular, intrapulmonary, intracranial, intraventricular, intraspinal, dermal, subdermal, intra-articular, placement within cavities of the body, nasal inhalation, pulmonary inhalation, impression into skin, and electroporation. Administration can be systemic or local. Pharmaceutical compositions can be delivered locally to the area in need of treatment, for example by topical application or local injection. Multiple administrations and/or dosages can also be used. Effective doses can be extrapolated from dose-response curves derived from in vitro or animal model test systems. For in vitro and ex vivo administration, the effective doses for the agent(s) can be determined with cell cultures or biological samples and dose response curves.

In an example in which a nucleic acid is employed, such as, an antisense, a morpholino, an siRNA molecule, or a locked nucleic acid, the nucleic acid can be delivered intracellularly (for example by expression from a nucleic acid vector or by receptor-mediated mechanisms), or by an appropriate nucleic acid expression vector which is administered so that it becomes intracellular, for example by use of a retroviral vector (see U.S. Pat. No. 4,980,286), or by direct injection, or by use of microparticle bombardment (such as a gene gun; Biolistic, Dupont), or coating with lipids or cell-surface receptors or transfecting agents, or by administering it in linkage to a homeobox-like peptide which is known to enter the nucleus (for example Joliot et al., Proc. Natl. Acad. Sci. USA 1991, 88:1864-8). Nucleic acid carriers also include, polyethylene glycol (PEG), PEG-liposomes, branched carriers composed of histidine and lysine (HK polymers), chitosan-thiamine pyrophosphate carriers, surfactants, nanochitosan carriers, and D5W solution. Physical transduction techniques can also be used, such as liposome delivery and receptor-mediated and other endocytosis mechanisms (see, for example, Schwartzenberger et al., Blood 87:472-478, 1996) to name a few examples. This invention can be used in conjunction with any of these or other commonly used gene transfer methods. The present disclosure includes all forms of nucleic acid delivery, including naked DNA, plasmid and viral delivery, integrated into the genome or not.

As mentioned above, vector delivery can be via a viral system, such as a retroviral vector system which can package a recombinant retroviral genome (see e.g., Pastan et al., Proc. Natl. Acad. Sci. U.S.A. 85:4486, 1988; Miller et al., Mol. Cell. Biol. 6:2895, 1986). The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naidini et al., Science 272:263-267, 1996), and pseudotyped retroviral vectors (Agrawal et al., Exper. Hematol. 24:738-747, 1996).

The disclosure also provides a pharmaceutical pack or kit comprising one or more containers filled with one or more of the ingredients of the pharmaceutical compositions (e.g., NMD inhibitors, chemotherapeutic agents, and carriers). Optionally associated with such container(s) can be a notice in the form prescribed by a governmental agency regulating the manufacture, use or sale of pharmaceuticals or biological products, which notice reflects approval by the agency of manufacture, use or sale for human administration. Instructions for use of the composition can also be included.

Depending on the intended mode of in vivo administration, the pharmaceutical composition can be in the form of solid, semi-solid, or liquid dosage forms, optionally in unit dosage form suitable for single administration of a precise dosage. The compositions will include a therapeutically effective amount of the agent(s) described herein or derivatives thereof in combination with a pharmaceutically acceptable carrier and, in addition, can include other medicinal agents, pharmaceutical agents, carriers, or diluents. By pharmaceutically acceptable is meant a material that is not biologically or otherwise undesirable, which can be administered to an individual along with the selected agent(s) without causing unacceptable biological effects or interacting in a deleterious manner with the other components of the pharmaceutical composition in which it is contained.

Compositions containing one or more of the agents described herein or pharmaceutically acceptable salts or prodrugs thereof suitable for parenteral injection can comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions can also contain adjuvants such as preserving, wetting, emulsifying, and dispensing agents. Prevention of the action of microorganisms can be promoted by various antibacterial and antifungal agents, for example, parabens, chlorobutanol, phenol, sorbic acid, and the like. Isotonic agents, for example, sugars, sodium chloride, and the like can also be included. Prolonged absorption of the injectable pharmaceutical form can be brought about by the use of agents delaying absorption, for example, aluminum monostearate and gelatin.

Solid dosage forms for oral administration of the agents described herein or pharmaceutically acceptable salts or prodrugs thereof include capsules, tablets, pills, powders, and granules. In such solid dosage forms, the agents described herein or derivatives thereof is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms can also comprise buffering agents.

Solid compositions of a similar type can also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others known in the art. They can contain opacifying agents and can also be of such composition that they release the active agent(s) in a certain part of the intestinal tract in a delayed manner. Examples of embedding compositions that can be used are polymeric substances and waxes. The active agents can also be in micro-encapsulated form, if appropriate, with one or more of the above-mentioned excipients.

Liquid dosage forms for oral administration of the agents described herein or pharmaceutically acceptable salts or prodrugs thereof include pharmaceutically acceptable emulsions, solutions, suspensions, syrups, and elixirs. In addition to the active agents, the liquid dosage forms can contain inert diluents commonly used in the art, such as water or other solvents, solubilizing agents, and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like.

Besides such inert diluents, the composition can also include additional agents, such as wetting, emulsifying, suspending, sweetening, flavoring, or perfuming agents.

Suspensions, in addition to the active agents, can contain additional agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these agents may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Throughout this application, various publications are referenced. The disclosures of these publications in their entireties are hereby incorporated by reference into this application.

A number of aspects have been described. Nevertheless, it will be understood that various modifications can be made. Furthermore, when one characteristic or step is described it can be combined with any other characteristic or step herein even if the combination is not explicitly stated. Accordingly, other aspects are within the scope of the claims.

The following examples are put forth so as to provide those of ordinary skill in the art with a complete disclosure and description of how the agents, compositions and/or methods claimed herein are made and evaluated, and are intended to be purely exemplary of the invention and are not intended to limit the scope of what the inventors regard as their invention except as and to the extent that they are included in the accompanying claims.

### Examples

### Reagents

Doxorubicin (Sigma, St. Louis, Mo.; D1515), cycloheximide (Sigma; C4859), etoposide (Sigma; E1383) staurosporine (EMD; Temecula, Calif.); 569397), caspase inhibitors (EMD; set IV 80510-354), human TNF-α (Invitrogen, Carlsbad, Calif.; PHC3015), N-ethyl maleimide (NEM; Sigma; E3876), DRB (Sigma; D1916), and doxycycline (Sigma; D3072) were used at the concentrations and times indicated in figures and figure legends. NMDI-1 was a gift. Active recombinant Caspase 3 and 7 were from PromoKine (Heidelberg, Germany). HeLa Tet-off cells were obtained from Clontech (Mountain View, Calif.).

### Cell Culture and Transfections

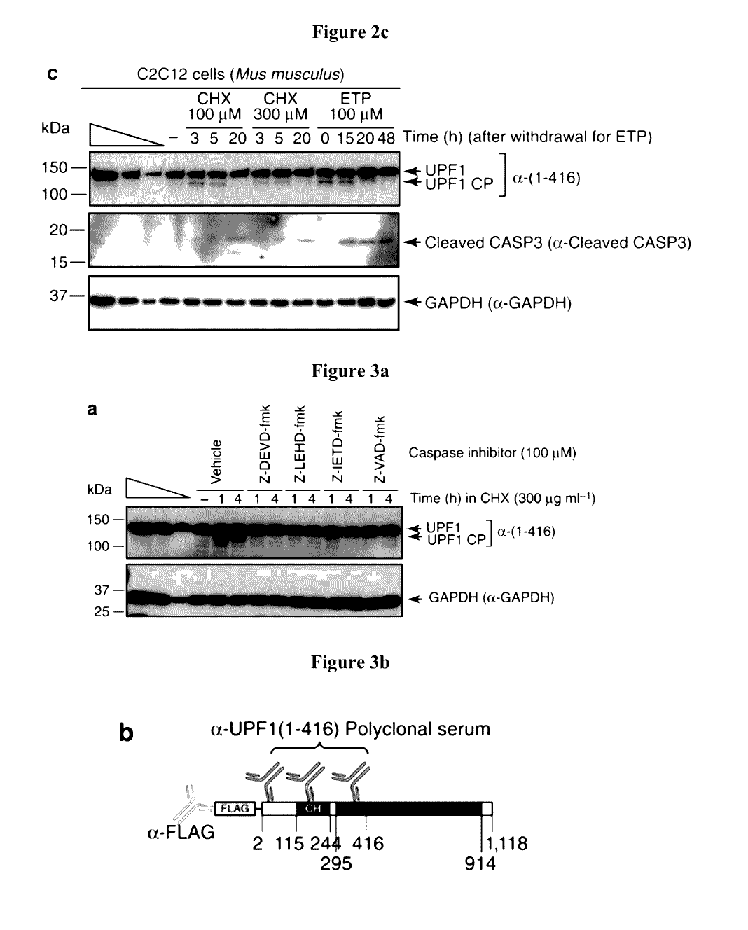

All cell lines were cultivated in DMEM (Gibco, Waltham, Mass.) containing 10% fetal bovine serum (Gibco) with the exception of Jurkat and Daudi cells, which were grown in RPMI-1640 (Gibco) with 10% fetal bovine serum. HeLa and MCF7 cells (ATCC) were transfected with plasmid DNA using Lipofectamine LTX (Invitrogen), and HEK293T cells were transfected with plasmid DNA using Lipofectamine 2000 (Invitrogen). Transfections using siRNA employed RNAi MAX (Invitrogen) according to manufacturer's directions, with the exception of FIG. 2a, which employed Oligofectamine (Invitrogen). Cells were plated in antibiotic-free medium for a minimum of 24 h before transfection and harvested 48 h after plasmid transfections or 72 h after siRNA transfections.

### Western Blotting

Cells were lysed, and protein was isolated using hypotonic buffer that consists of 10 mM Tris-C1, pH 7.4, 10 mM NaCl, 2 mM EDTA, 0.5% Triton X-100, 2 mM benzamidine, 1 mM PMSF, NEM (50 μg/mL), 1× phosphatase inhibitor cocktail (Roche), and 1× protease inhibitor cocktail (Roche; Basel, Switzerland). After 10 minutes of incubation at 4° C., NaCl was added to 150 mM, and lysates were cleared by centrifugation. Proteins were resolved using SDS-PAGE, transferred to Hybond ECL nitrocellulose (GE), and probed using antibody that recognizes one of the following: UPF1(1-416) (1:2000⁸), p-UPF1 S1116 (1:1000; Millipore 07-1016; Billerica, Mass.), p-UPF1 S1089 (1:1000; Millipore 07-1015), Calnexin (1:2000; Enzo Life Sciences ADISPA860; Farmingdale, N.Y.), UPF2 (1:200; Santa Cruz Biotechnology C18 20227; Dallas, Tex.), UPF3× and 3 (1:1000⁸), MLN51 (1:1000; Bethyl Laboratories A302-471; Montgomery, Tex.), GAPDH (1:200; Santa Cruz Biotechnology 25778), CBP80 (1:1000; Bethyl Laboratories A301-793), SMG5 (1:1000; Abcam 33033; Cambridge, United Kingdom), SMG6 (1:1000; Abcam 57539), SMG7 (1:1000; Bethyl Laboratories 302-170A), SMG1 (1:1000; Cell Signaling D42D5; Danvers, Mass.), Cleaved Caspase 3 (1:1000; Cell Signaling 9664), Cleaved Caspase 9 (1:1000; Cell Signaling 7237), PARP (1:1000; Cell Signaling 9542), Cleaved PARP (1:200; Santa Cruz Biotechnology sc56196) PLC-γ1 (1:200; Santa Cruz Biotechnology 58407), α-Tubulin (1:1000; Cell Signaling 3873P), FLAG (1:5000; Sigma, clone M2 a5982), HA (1:5000; Roche, clone 3F10 12013819001), or MYC (1:1000; Cell Signaling, clone 9B110 2276). Immunoreactivity was assessed using SuperSignal West Pico or Femto (Pierce Biotechnology; Waltham, Mass.).

siRNA Sequences

siRNAs used were control siRNA #3 (Ambion) and UPF1 siRNA (Thermo Fisher Scientific (Waltham, Mass.); 5′-GAUGCAGUUCCGCUCCAUUdTdT-3′) (SEQ ID NO: 1).

### Plasmid Constructions

All constructs were sequence-verified. pMYC-UPF1 dNT was described in Kashima et al. (Genes Dev. 20: 355-67 (2006)). pMYC-UPF1 R843C was described in Sun et al. (Proc. Natl. Acad. Sci. USA 95: 10009-14 (1998)). MYC-UPF1-FLAG WT cDNA, resistant to the siRNA below, was PCR-amplified (KOD polymerase, Novagen; Darmstadt, Germany) from pMYC-UPF1 (Gong et al. Genes Dev. 23: 54-66 (2009)) using the primer pair 5′-ATATGCAGATCTGCCACCATGGAGCAGAAGCTGATCTCAGAGGAGGACC-3′ (sense) (SEQ ID NO: 2) and 5′-ATCGATATCGATTTACTTATCGTCGTCATCCTTGTAATCGCCACCTGATCCGCCATAC TGGGACAGCCCCGTCA-3′ (SEQ ID NO: 3)(antisense, which encodes a FLAG-tag fused to the C-terminus of UPF1). The PCR product was cleaved with BglII and ClaI and inserted into BglII- and ClaI cleaved retroviral pLNCX2 (Clontech).

Δ17-UPF1-FLAG, Δ27-UPF1-FLAG, Δ37-UPF1-FLAG, Δ75-UPF1-FLAG fragments were PCR-amplified from MYC-UPF1-FLAG WT using the sense primer 5′-GCATGCATAGATCT GCCACCATGACGGAGGAGGCCGAGCT-3′ (SEQ ID NO: 4), 5′-GCATGCATAGATCTGCCACCATGACACAGGGCTCCGAGTTCGAG-3′ (SEQ ID NO: 5), 5′-GCATGCATAGATCTGCCACCATGTTTACTCTTCCTAGCCAGACGCAGACG-3′ (SEQ ID NO: 6), or 5′-GCATGCATAGATCTGCCACCATGGCGCAGGTTGGGCCC-3′ (SEQ ID NO: 7), respectively, and the antisense primer described for the MYC-UPF1-FLAG WT PCR product. Deletion fragments were inserted into pLNCX2 as described for MYC-UPF1-FLAG WT.

Plasmids expressing UPF1 variant MYC-UPF1-FLAG D37N or MYC-UPF1-FLAG TEV were generated using standard overlap-extension PCR (KOD polymerase, Novagen). For each, 5′ and 3′ PCR fragments were amplified using the primers listed below, gel-purified, mixed, and re-amplified using the sense primer for the 5′ fragment and the antisense primer for the 3′ fragment to obtain cDNA for full-length variant UPF1. cDNAs were then cleaved and inserted into cleaved retroviral pLNCX2 as described above for MYC-UPF1-FLAG WT cDNA.

MYC-UPF1-FLAG D37N 5′ fragment primer set:
(SEQ ID NO: 8)
5′-
ATATGCAGATCTGCCACCATGGAGCAGAAGCTGATCTCAGAGGAGGACC-
3′ (sense);
(SEQ ID NO: 9)
5′-AGGAAGAGTAAAGTTGGTGAACTCGAA-3′ (antisense).
MYC-UPF1-FLAG D37N 3′ fragment primer set:
(SEQ ID NO: 10)
5′-TTCGAGTTCACCAACTTTACTCTT CCT-3′ (sense);
(SEQ ID NO: 11)
5′-
ATCGATATCGATTTACTTATCGTCGTCATCCTTGTAATCGCCACCTGATC
CGCCATACTGGGACAGCCCCGTCA-3′ (antisense)
MYC-UPF1-FLAG TEV 5′ fragment primer set:
(SEQ ID NO: 12)
5′-
ATATGCAGATCTGCCACCATGGAGCAGAAGCTGATCTCAGAGGAGGACC-
3′ (sense);
(SEQ ID NO: 13)
5′-
GCTAGGAAGAGTAAAGGACTGGAAGTAGAGATTTTCGGTGAACTCGAACT
CGGAGCC-3′ (antisense)
MYC-UPF1-FLAG TEV 3′ fragment primer set:
(SEQ ID NO: 14)
5′-
GAAAATCTCTACTTCCAGTCCTTTACTCTTCCTAGCCAGACGCAGACG-
3′ (sense);
(SEQ ID NO: 15)
5′-
ATCGATATCGATTTACTTATCGTCGTCATCCTTGTAATCGCCACCTGATC
CGCCATACTGGGACAGCCCCGTCA-3′ (antisense).

Plasmid expressing β-Gl Norm, β-Gl 39 Ter, MUP, GPx1 Norm, or GPx1 46 Ter mRNAs were described in Kurosaki et al (Proc. Natl. Acad. Sci. USA (2013)).

3×FLAG-mCherry TCRβ reporter plasmids were generated as follows. A custom lentiviral vector harboring the bidirectional promoter from pBI-CMV1 (Clontech) was constructed using the pLVX-Puro vector (Clontech). First, the pBI bidirectional promoter region was excised by cleavage with SspI and SalI and inserted into pLVX-Puro that had been digested with ClaI, blunted with Klenow fragment (New England Biolabs), and further digested with XhoI. To eliminate an ApaI site in the pLVX-Puro backbone, the resulting vector was digested with BstbI, blunted using Klenow, and the linearized vector was further digested using SmaI. The resultant linear fragment was circularized using T4 ligase. Next, the puromycin-resistance cassette was removed, and Zeocin resistance was substituted. The Zeocin-resistance cassette was PCR-amplified from the pcDNA3.1 Zeo vector (Invitrogen) using the primer set 5′-GCATCGATCGATGCCACCATGGCCAAGTTGACCAGTGCC-3′ (sense) (SEQ ID NO: 16) and 5′-CTGACTGGTACCTCAGTCCTGCTCCTCGGC-3′ (antisense) (SEQ ID NO: 17), and digested with ClaI and KpnI. A PGK promoter fragment was amplified from pLVX-PURO using the primer set 5′-CGTCTCACTAGTCTCGTGCAGATGG-3′ (sense) (SEQ ID NO: 18) and 5′-ACTGCTATCGATCTTGGGCTGCAGGTCGAAAGG-3′ (antisense) (SEQ ID NO: 19), digested using SpeI and ClaI, and both the PGK promoter fragment and Zeocin-resistance cassette fragment were simultaneously ligated into SpeI- and KpnI-digested bidirectional lentiviral vector. Finally, a Woodchuck Hepatitis Virus Posttranscriptional Regulatory Element (WPRE) fragment was amplified from pLVX-PURO using the primer set 5′-GCATGCGGTACCCCGCGTCTGGAACAATCAACCTC-3′ (sense) (SEQ ID NO: 20) and 5′-ACTCGACTGGTACCTGAGGTGTGACTGGAAAACC-3′ (antisense) (SEQ ID NO: 21), digested with KpnI, and inserted into similarly digested vector to yield the final lentiviral vector harboring a bidirectional promoter.

Next, HA-tagged mCerulean cDNA was PCR-amplified from the plasmid mCerulean-C1 (Clontech) using the primer set 5′-ATGCATACGCGTGCCACCATGTACCCATACGATGTTCCAGATTACGCTGGCGGAGGT ATGGTGAGCAAGGGCGAGG-3′ (sense) (SEQ ID NO: 22) and 5′-ATGCATGCGGCCGCTCACTTGTACAGCTCGTCCATGCCG-3′ (antisense) (SEQ ID NO: 23), digested with MluI and NotI, and inserted into similarly digested bidirectional lentiviral vector. Reporters encoding 3× FLAG mCherry-TCRβ ΔJC intron and 3× FLAG mCherry-TCRβ+JC intron were generated. Briefly, 3× FLAG-mCherry was PCR-amplified from pmCherry-C1 (Clontech) using the primer set 5′-AAAGATCATGACATCGATTACAAGGATGACGATGACAAGGGCGGAGGTATGGTGAG CA AG-3′ (sense) (SEQ ID NO: 24) and 5′-CGTATAATGTATGCTATACGAAGTTATATCAGTCAGTCACTTGTACAGCTCGTCCAG CC-3′ (antisense) (SEQ ID NO: 25). The resultant PCR product was subjected to a second round of PCR using the primers set 5′-GCATGCATGGGCCCGCCACCATGGACTACAAAGACCATGACGGTGATTAT AAAGATCATGACATCGATTACAAGGATGACGATG-3′ (sense) (SEQ ID NO: 26) and 5′-GTACCCATCAGGGATATCTCCTTTCTCCGTGCTGTCAGCGACATAACTTCGTATAATG TATGCTATACGAAGTTATATCAGTCAGT-3′ (antisense) (SEQ ID NO: 27) to obtain the 3× FLAG-mCherry fragment. The +JC intron and ΔJC intron 3′UTR fragments were amplified from the plasmids using the primer set 5′-CGGAGAAAGGAGAT ATCCCTGATGGGTAC-3′ (sense) (SEQ ID NO: 28) and 5′-CTTCGTATAATGTATGCTATACGAAGTTATTCAACGAGGAAGGTGGTCAGGG-3′ (antisense) (SEQ ID NO: 29), and each fragment was used as template in a second round of PCR using the primer set 5′-CGGAGAAAGGAGATATCCCTGATGGGTAC-3′ (sense) (SEQ ID NO: 30) and 5′-GCATGCGGGCCCATAACTTCGTATAATGTATGCTATACGAAGTTATTCAACGAGG-3′ (antisense) (SEQ ID NO: 31). The resultant fragments were fused to the 3× FLAG-mCherry fragment using standard overlap extension PCR and the primer set 5′-GCATGCATGGGCCCGCCACCATGGACTACAAAGACCATGACGGTGATTAT AAAGATCATGACATCGATTACAAGGATGACGATG-3′ (sense) (SEQ ID NO: 32) and 5′-GCATGCGGGCCCATAACTTCGTATAATGTATGCTATACGAAGTTATTCAACGAGG-3′ (antisense) (SEQ ID NO: 33).

PCR products were digested with ApaI and inserted into similarly digested bidirectional lentiviral vector containing HA-mCerulean to yield the final reporter plasmids. A bidirectional promoter plasmid simultaneously encoding MYC-NTEV and MYCCTEV fragments was generated by amplifying MYC-NTEV cDNA from the plasmid using the primer set 5′-CGATCGGATCCGCCACCATGGAACAAAAGCTGATCTCTG-3′ (sense) (SEQ ID NO: 34) and 5′-GATCATGCGGCCGCTCAGGTCTGGAAGTTGGTGGTC-3′ (antisense) (SEQ ID NO: 35). The resulting PCR fragment was digested using BamHI and NotI, and inserted into similarly digested pBI-CMV1 (Clontech). MYC-CTEV cDNA was next amplified using the primer set 5′-GGAATTAGATCTGCCACCATGGAACAAAAGCTGATCTCTG-3′ (sense) (SEQ ID NO: 36) and 5′-AATTCCTCTAGATCACATGAACACCTTATGTCCGCC-3′ (antisense) (SEQ ID NO: 37), digested using BglII and XbaI, and inserted into similarly digested plasmid to obtain the final construct. Human β-Gl Norm and β-Gl 39 Ter genes inserted into the pcTET2 vector were obtained from Jens Lykke-Andersen.

### RT-qPCR

Cells were homogenized, and RNA was purified using RNeasy mini kits (Qiagen). To ensure linearity of both reverse transcription (RT) and qPCR analyses, 17 standard concentrations were constructed using two-fold serial dilutions of total-cell RNA, beginning with 2 μg. Experimental sample input was 200 ng of RNA. RT was conducted using Superscript III (Invitrogen) according to manufacturer's directions and 100 ng of random hexamer (Invitrogen) in a 20 μl reaction volume. Thermal cycling was as follows: 25° C. for 5 min; 42° C. for 60 min; 50° C. for 60 min; and 75° C. for 10 min. Reactions were diluted to a final volume of 80 μl with water and used for qPCR.

qPCR was conducted using FAST SYBR green master mix (ABI; Foster City, Calif.) according to manufacturer's directions. Cycling was done using a 7500 FAST Real Time machine (ABI) with default conditions, and thermal denaturation curves were generated for each run. Primer sequences were as follows, with mRNA primers sets containing at least one primer spanning an exon-exon junction, and pre-mRNA primer sets containing at least one primer that hybridizes to intronic sequences. Primers for Gl mRNA, GPx1 mRNA and MUP mRNA were described in Kurosaki et al.

GAPDH mRNA:
(SEQ ID NO: 38)
5′-GTCGCCAGCCGAGCCACATC-3′ (sense);
(SEQ ID NO: 39)
5′-CCAGGCGCCCAATACGACCA-3′ (antisense).
GADD45α mRNA:
(SEQ ID NO: 40)
5′-GGAGGAATTCTCGGCTGGAG-3′ (sense);
(SEQ ID NO: 41)
5′-CGTTATCGGGGTCGACGTT-3′ (antisense).
GADD45 α pre-mRNA:
(SEQ ID NO: 42)
5′-TTGCAGGGAACCCAACTACC-3′ (sense);
(SEQ ID NO: 43)
5′-TCCTTCCATTGAGATGAATGTGG-3′ (antisense).
GADD45β mRNA:
(SEQ ID NO: 44)
5′-TTGTCTCCTGGTCACGAACC-3′ (sense);
(SEQ ID NO: 45)
5′-TGTGGCAGCAACTCAACAGA-3′ (antisense).
GADD45 pre-mRNA:
(SEQ ID NO: 46)
5′-GATGAATGTGTGAGTCAGACC-3′ (sense);
(SEQ ID NO: 47)
5′-GCAGACGATACATCAGGATAC-3′ (antisense).
CDKN1A mRNA:
(SEQ ID NO: 48)
5′-TGGAGACTCTCAGGGTCGAA-3′ (sense);
(SEQ ID NO: 49)
5′-GGATTAGGGCTTCCTCTTGG-3′ (antisense).
CDKN1A pre-mRNA:
(SEQ ID NO: 50)
5′-CCAGGGCCTTCCTTGTATCT-3′ (sense);
(SEQ ID NO: 51)
5′-GCATGGGTTCTGACGGAC-3′ (antisense).
mCherry mRNA:
(SEQ ID NO: 52)
5′-TTCATGTACGGCTCCAAGGC-3′ (sense);
(SEQ ID NO: 53)
5′-TGTAGATGAACTCGCCGTCC-3′ (antisense).
Cerulean mRNA:
(SEQ ID NO: 54)
5′-GGACGACGGCAACTACAAGA-3′ (sense);
(SEQ ID NO: 55)
5′-TTGTCGCTGATGGCGTTGTA-3′ (antisense).
GAS5 non-coding mature (spliced) RNA:
(SEQ ID NO: 56)
5′-CTTGCCTGGACCAGCTTAAT-3′ (sense);
(SEQ ID NO: 57)
5′-CAAGCCGACTCTCCATACCT-3′ (antisense).
BAK1 mRNA:
(SEQ ID NO: 58)
5′-ACCTGAAAAATGGCTTCGG-3′ (sense);
(SEQ ID NO: 59)
5′-GTAGCTGCGGAAAACCTCCT-3′ (antisense).
DAP3 mRNA:
(SEQ ID NO: 60)
5′-GCAAAACAGGACTGGCTGAT-3′ (sense);
(SEQ ID NO: 61)
5′-ACTGCAGAAGATCCCGACAA-3′ (antisense).
DUSP2 mRNA:
(SEQ ID NO: 62)
5′-GGCCTTTGACTTCGTTAAGC-3′ (sense);
(SEQ ID NO: 63)
5′-CCACCTCAGTGACACAGCAC-3′ (antisense).
BCL3 mRNA:
(SEQ ID NO: 64)
5′-CACCCGTGCAGATGAGGA-3′ (sense);
(SEQ ID NO: 65)
5′-CTGCTGGAAGAGGTTGACCA-3′ (antisense).
hβG1 mRNA:
(SEQ ID NO: 66)
5′-ACTTCAGGCTCCTGGGCAAC-3′ (sense);
(SEQ ID NO: 67)
5′-CAGCAAGAAAGCGAGCTTAGTG-3′ (antisense).
β-ACTIN mRNA:
(SEQ ID NO: 68)
5′-AATCGTGCGTGACATTAAG-3′ (sense);
(SEQ ID NO: 69)
5′-ATGATGGAGTTGAAGGTAGT-3′ (antisense).
TSTD2 mRNA:
(SEQ ID NO: 70)
5′-CATGCTTTCCTTCCCATTGTT-3′ (sense);
(SEQ ID NO: 71)
5′-ATGGGCACGATTTCTTCAAA-3′ (antisense).
TSTD2 pre-mRNA:
(SEQ ID NO: 72)
5′-CTGGAGATGGGACAGCAAGA-3′ (sense);
(SEQ ID NO: 73)
5′-ATGGGCACGATTTCTTCAAA-3′ (antisense).
PANK2 mRNA:
(SEQ ID NO: 74)
5′-GGGATATAGACGGGAGCCAT-3′ (sense);
(SEQ ID NO: 75)
5′-CCACCGATATCCAGTCCAAA-3′ (antisense).
PANK2 pre-mRNA:
(SEQ ID NO: 76)
5′-CCCTAGCGTTTGAAATAAGTTGC-3′ (sense);
(SEQ ID NO: 77)
5′-CCACCGATATCCAGTCCAAA-3′ (antisense).
NAT9 mRNA:
(SEQ ID NO: 78)
5′-ATTGTGCTGGATGCCGAGA-3′ (sense);
(SEQ ID NO: 79)
5′-ACCTAGCGTGGTCACTCCGTA-3′ (antisense).
NAT9 pre-mRNA:
(SEQ ID NO: 80)
5′-TGTCTAAAGCCACCCCTCTG-3′ (sense);
(SEQ ID NO: 81)
5′-CTGCATGGCATACTCCTGCT-3′ (antisense).
18S rRNA:
(SEQ ID NO: 82)
5′-GGGAAACCAAAGTCTTTGGG-3′ (sense);
(SEQ ID NO: 83)
5′-GGAATTAACCAGACAAATCGC-3′ (antisense).

### Stable Cell-Line Generation

Stable cell lines were generated using retroviral transduction as described in Esteban et al. (Proc. Natl. Acad. Sci. USA 108: 14270-5 (2011)). Cells were transduced at a multiplicity of infection of ˜0.1 to ensure single copy integration. HeLa cells were selected in 800 μg/mL Geneticin (Gibco). Before subsequent experiments, selected cells were cultured in geneticin-free medium for at least two days prior to use.

Immunoprecipitation and on-Bead RNase Digestion

HEK293T cells were transfected as described in the figure legends. Cells were lysed as described for Western blotting. Input lysate protein concentrations were determined using the Bradford method (Biorad; Hercules, Calif.), equalized, and pre-cleared twice using protein-A conjugate agarose (Roche) for 30 min with end-over-end rotation at 4° C. Pre-cleared lysates were subjected to immunoprecipitation (IP) using anti-FLAG M2 Sepharose (Sigma) for 2 h at 4° C., washed with lysis buffer supplemented to contain 0.1% TritonX-100, and divided into two equal volumes. One-half of each IP was incubated with BSA in RNAse ONE (Promega; Madison, Wis.) reaction buffer, and the other half was incubated with 1000 U RNAse ONE (Promega) in reaction buffer for 30 min at 4° C. Samples were washed three times with wash buffer and eluted using 3× FLAG peptide (Sigma) according to manufacturer's directions.

### In-Vitro Caspase Cleavage Assays

HEK293T cells were transfected with the indicated constructs. Cells were harvested 48 h later, and anti-FLAG immunoprecipitation was performed as above, without RNase ONE digestion. Proteins were eluted with 3× FLAG peptide. One microliter of each immunoprecipitate was incubated for 5 h at 37° C. with 6 U of either Caspase 3 or Caspase 7 in caspase cleavage buffer (50 mM HEPES pH 7.2, 50 mM NaCl, 0.1% CHAPS, 10 mM EDTA, 5% glycerol, and 10 mM DTT).

mRNA Decay Assays

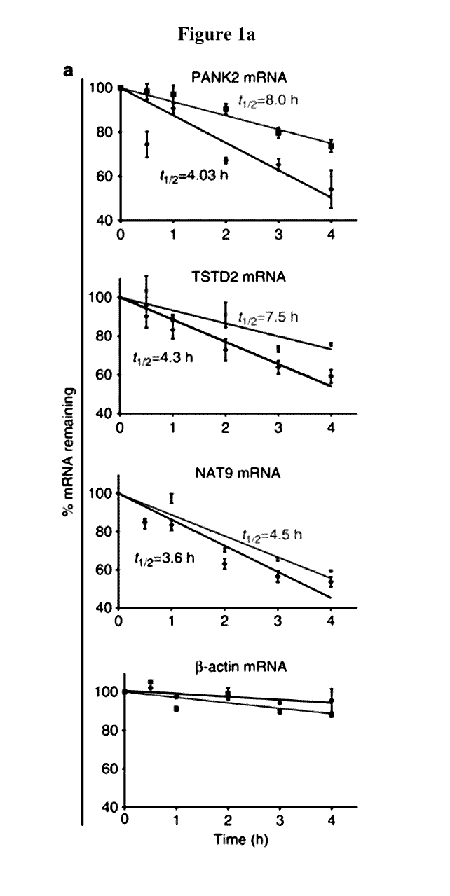

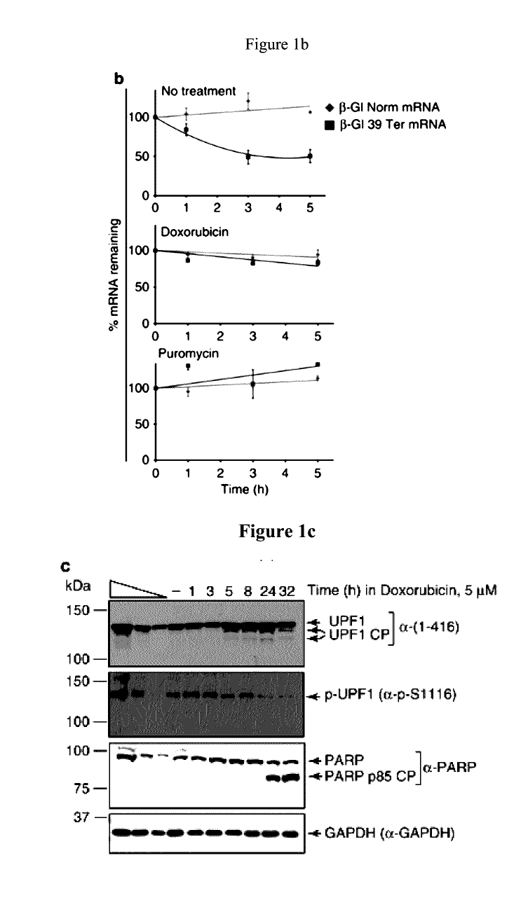

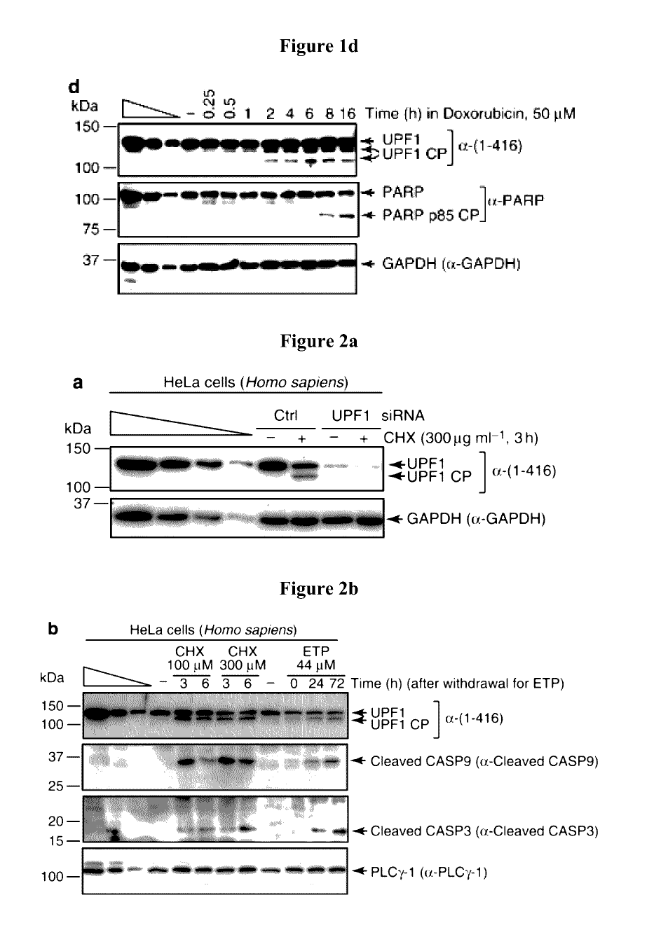

For mRNA decay assays using actionmycin D, MCF7 cells were plated at 103,000 cells/well in 24-well dishes. After 24 h, cells either were or were not pre-treated for 1 h with 5 μM doxorubicin before addition of 3 μg/ml actinomycin D (Sigma). Cells were harvested at the indicated time points. Doxorubicin-treated cells received doxorubucin during the chase period. For Tet-off assays, HeLa Tet-off cells (Clontech) were plated at 40,000 cells/well in 24-well dishes. After 16 h, cells were transfected with the indicated plasmids in the presence of 1 μg/ml doxycycline to inhibit transcription. After 48 h, cells were washed three times with medium lacking doxycycline and incubated for 5 h without doxycycline to induce transcription. Cells were then either treated with nothing, treated with 50 μM doxorubicin for 1 h prior to transcriptional shut-off, or treated with 50 μg/ml puromycin for 3 h prior to transcriptional shutoff. At t=0, cells were cultured in medium containing 2 μg/ml doxycycline to induce shut-off and subsequently harvested at the indicated time points. For cells treated with doxorubicin or puromycin, these compounds were included in the chase. RT-qPCR was used to assess mRNA levels during the chase time. Data for actinomycin D decay assays (FIG. 1a) as well as for Tet-off decay assays (FIG. 1b), with the exception of the no treatment 0-Gl 39 Ter mRNA data, were fitted to best fit linear regression lines because they clearly exhibited single component decay kinetics. In contrast, the no treatment 0-Gl 39 Ter mRNA has been shown to decay with two-component kinetics. For DRB-treated mRNA decay assays, HeLa cells were transfected with the indicated plasmids using Lipofectamine LTX. After 48 h, cells were treated with 100 μg/ml DRB (Sigma). Cells were harvested at the indicated time points, and RT-qPCR was used to assess transcript levels.

### TEV Cleavage and RNA-Seq

For the TEV cleavage experiments, stably transduced HeLa cell lines were plated at 200,000 cells/well in a 6-well dish and cultured without antibiotics for 48 h. Either the bidirectional TEV protease-encoding plasmid or empty vector was introduced, and cells were harvested and flash frozen 16 h later. RNA was processed as for RT-qPCR. Biological duplicates of each sample (each duplicate consisted of 6 pooled wells) were used for sequencing. Total-cell RNA was submitted to the Whitehead Institute Genome Technology Core for RNA-seq. RNA concentrations were determined using a NanopDrop 1000 spectrophotometer (NanoDrop, Wilmington, Del.), and RNA quality was assessed using a Agilent Bioanalyzer (Agilent, Santa Clara, Calif.). Poly(A)⁺ libraries were prepared using the automated IntegenX Apollo system. Sequencing was performed using an Illumina HiSeq 2500 in 40-bp single-read mode. Data analysis was performed by the University of Rochester Genomics Research Center. Raw reads generated from the Illumina HiSeq2500 sequencer were demultiplexed using configurebcl2fastq.pl version 1.8.3. Low complexity reads and vector contamination were removed using sequence cleaner (“seqclean”) and the NCBI univec database, respectively. The FASTX toolkit (fastq quality trimmer) was applied to remove bases with quality scores below Q=13 from the end of each read. Processed reads were then mapped to the UCSC Hg19 genome build using SHRiMP version 2.2.3, and differential expression analysis was performed using Cufflinks version 2.0.2; specifically, cuffdiff2 and usage of the general transfer format (GTF) annotation file for the given reference genome. Pooled duplicate values were used for analyses. Specifically, fold-change in Fragments Per Kilobase of transcript per Million (FPKM) values for cell lines stably transduced with empty vector and subsequently transiently transfected with TEV-encoding plasmid or empty vector were calculated. The same calculation was performed with FPKM values for the MYC-UPF1-FLAG TEV cell line. These values obtained for the MYC-UPF1-FLAG TEV cell line were then normalized to the fold-change value obtained for the empty vector stable cell line, and this calculation was performed for all genes represented.

### Cell Survival Assays

Cells were plated in white opaque 96-well plates using multichannel pipettors for accuracy and treated as described in the figure legends. CellTiter-Glo luminescent assays (Promega) were performed according to manufacturer's directions. Data were collected using a SpectraMax M2 plate reader.

### NMD Activity is Blunted During Doxorubicin Treatment

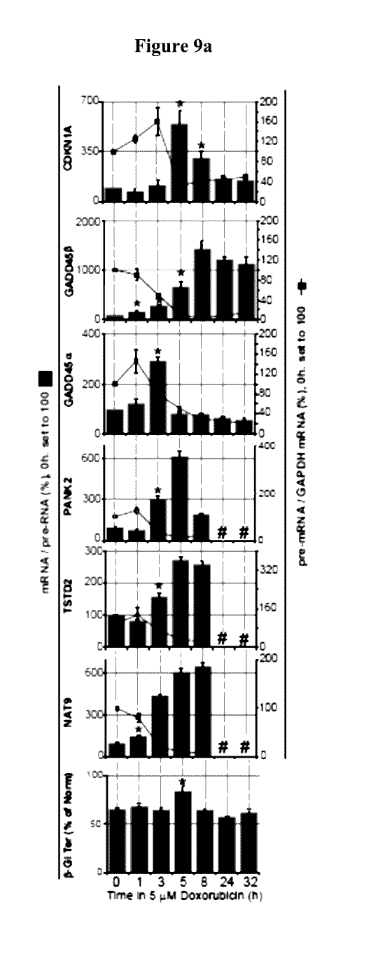

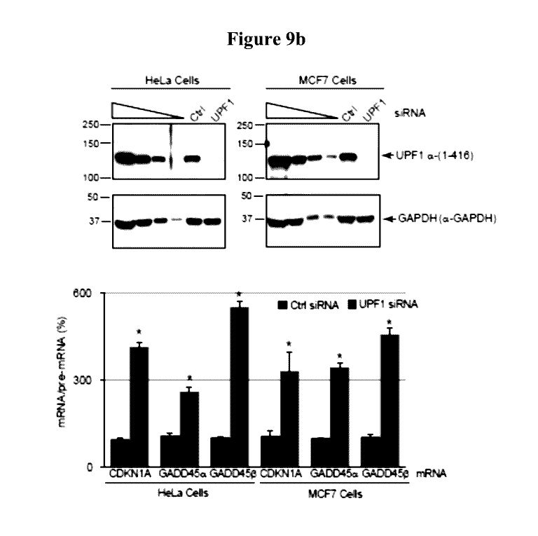

The stability of a panel of known NMD target mRNAs in human MCF7 breast cancer cells during doxorubicin treatment was examined. Pre-treatment with doxorubicin (5 μM) resulted in significant increases in the half-lives of PANK2, TSTD2, and NAT9 mRNAs but not β-actin mRNA after actinomycin D-mediated transcriptional arrest (FIG. 1a), indicating a decline in NMD activity. To support this, the level of each mRNA was measured relative to the level of the pre-mRNA from which it derives as a function of time after doxorubicin treatment to control for transcriptional effects. An increase in the mRNA/pre-mRNA ratio (a metric used to distinguish a subset of direct NMD targets from those that are not in UPF1-ablated HeLa cells) may not reliably distinguish NMD targets from those that are not during doxorubicin treatment, because global transcriptional shut-down may inflate this number. However, a decrease in this ratio would rule out the possibility that NMD activity is blunted. Consistent with the half-life data, the mRNA/pre-mRNA ratio, as assessed using RT-quantitative PCR (qPCR), increases for all three transcripts (none of which is known to be stress-regulated), in response to doxorubicin (FIG. 9a). The mRNA/pre-mRNA ratios for three additional known NMD-targeted transcripts, CDKN1A, GADD45α, and GADD45β, were also significantly increased by 5 hours (h) of doxorubicin treatment. As in HeLa cells, the ratio of CDKN1A, GADD45α, and GADD45β mRNAs to their corresponding pre-mRNAs is elevated upon UPF1 depletion in MCF7 cells, indicating that these mRNAs are indeed NMD targets in MCF7 cells (FIG. 9b). Decreases in pre-mRNA levels cannot account for the increased mRNA/pre-mRNA ratio since, even in the most extreme example (CDKN1A RNA), at 5 h the mRNA/pre-mRNA ratio increased ˜5.7 fold relative to 0 h, while the pre-mRNA level decreased only ˜3.2 fold.

To further corroborate the inhibition of NMD during doxorubicin treatment, MCF7 cells were transfected with the previously described 3-globin (β-Gl) NMD reporter plasmids encoding either β-Gl Norm transcripts that lack a PTC, or β-Gl Ter transcripts that harbor a PTC at position 39. Cells were cotransfected with a plasmid encoding the mouse urinary protein (MUP) transcript to control for variations in transfection efficiency and RNA recovery and, 24 h later, were exposed to doxorubicin (5 μM). By 5 h of doxorubicin treatment, the level of β-Gl Ter mRNA increased from ˜65% to ˜85% the level of β-Gl Norm mRNA. These measurements occur on the backdrop of global RNA degradation at later time points, accounting for why the normalized ratio of β-Gl Ter mRNA to β-Gl Norm mRNA is not elevated at later time points.

Additional mRNA decay assays were performed using a previously described HeLa Tet-off cell system to halt the synthesis of human β-Gl Norm mRNA or β-Gl Ter mRNA and subsequently measured the remaining levels of each mRNA relative to the level of MUP mRNA after doxycycline addition. This system was used because the Tet-off promoter that controls the production of β-Gl Norm mRNA or β-Gl Ter mRNA is not stress-responsive and HeLa cells, like MCF7 cells, are devoid of erythroid cell-specific β-Gl mRNA. Without doxorubicin, the level of β-Gl Ter mRNA declined to ˜50% of its starting level by ˜180 minutes of doxycycline addition, in agreement with reported values (Singh et al. “A competition between stimulators and antagonists of Upf complex recruitment governs human nonsense-mediated mRNA decay,” PLoS Biol. 2008; 6:e111), while the level of β-Gl Norm mRNA did not decrease during this time (FIG. 1b; top). In contrast, pretreatment of cells with doxorubicin for 1 h before doxycycline addition eliminated the selective decay of β-Gl Ter mRNA; the half-lives of both β-Gl Norm mRNA and β-Gl Ter mRNA exceeded the chase period (FIG. 1b; middle). Doxorubicin treatment mirrored the effect of the translational inhibitor puromycin, which is known to inhibit NMD (FIG. 1b; bottom). Based on these results, NMD activity is attenuated during doxorubicin treatment.

Biochemical changes to the key NMD factor, UPF1, that correlate with doxorubicin treatment (FIG. 1c), were examined. MCF7 cells were exposed to doxorubicin (5 μM) for varying amounts of time and cell lysates were analyzed using western blotting and an in-house generated polyclonal rabbit serum raised against the N-terminal 416 amino acids of human UPF1. To eliminate post-lysis proteolysis, lysates were generated in the presence of a protease inhibitor cocktail supplemented with N-ethylmaleimide at levels (50 μg/mL) known to alkylate the most active viral cysteine proteases. In addition to full-length UPF1, two additional bands of greater mobility were resolved by 5 h of doxorubicin treatment (FIG. 1c). Phosphorylation of UPF1 at both its N- and C-termini is a key feature that differentiates UPF1-bound NMD targets destined for degradation from those that are not. Western blotting using a monoclonal antibody recognizing phosphorylated S1116 revealed that UPF1 phosphorylation levels diminish by 5 h. Both of these changes to UPF1 preceded maximal cleavage of poly (ADP-ribose) polymerase (PARP), a well-characterized biochemical marker for apoptosis. Increasing doxorubicin concentrations ten-fold (50 μM) to accelerate apoptotic progression generated higher-mobility UPF1 species by 2 h, i.e., well before production of the PARP cleavage product at 8 h (FIG. 1d). Thus, the generation of faster-migrating UPF1 species, which were characterized as cleavage products (CPs), and the reduction of UPF1 phosphorylation occur early during apoptotic progression.

### Multiple Apoptotic Insults Cause UPF1 Hydrolysis

The upper UPF1 CP was characterized because it was consistently generated by an array of treatments in many cell lines (see below). This band derives from cellular UPF1 rather than a protein that fortuitously cross-reacts with the polyclonal anti-UPF1 serum, which was verified by using siRNA to reduce the level of UPF1 in human cervical carcinoma HeLa cells to <10% of normal and subsequently exposing cells to cycloheximide (CHX) to induce apoptosis (FIG. 2a). In addition to halting protein synthesis, CHX causes apoptosis via incompletely understood mechanisms. siRNA treatment reduced the levels of both full-length UPF1 and the UPF1 CP. Because new protein synthesis is halted by CHX, the UPF1 CP is unlikely to be a UPF1 isoform explained by the hypothetical possibility that alternative splicing of UPF1 pre-mRNA is induced during apoptosis.

Cleavage of proteins by caspases, a class of cysteine proteases, during apoptosis is a common event. “Bystander” cuts to proteins fortuitously encoding a caspase cleavage site may occur during apoptosis, but cleavage early during apoptotic progression and cleavage conservation across species indicate functional relevance.

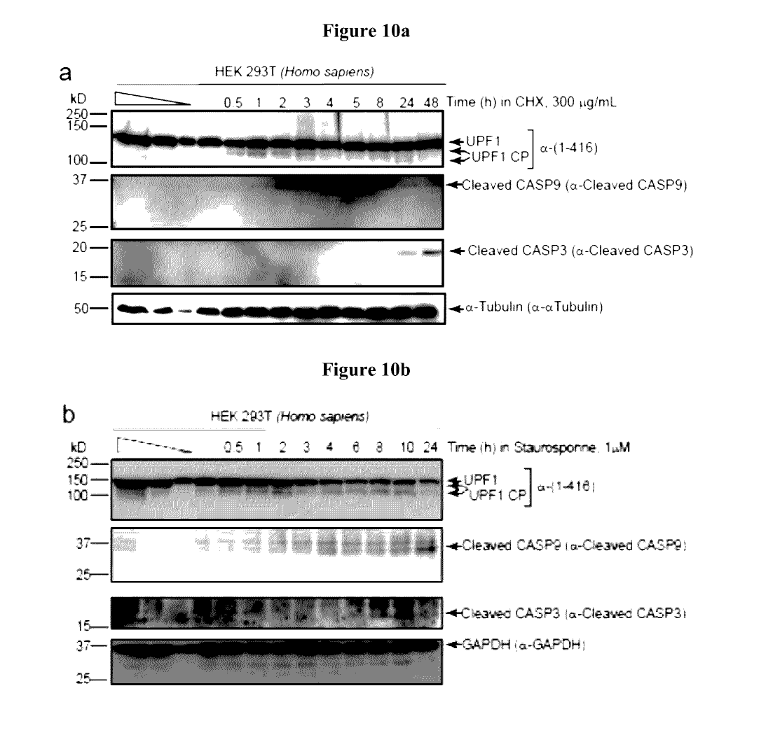

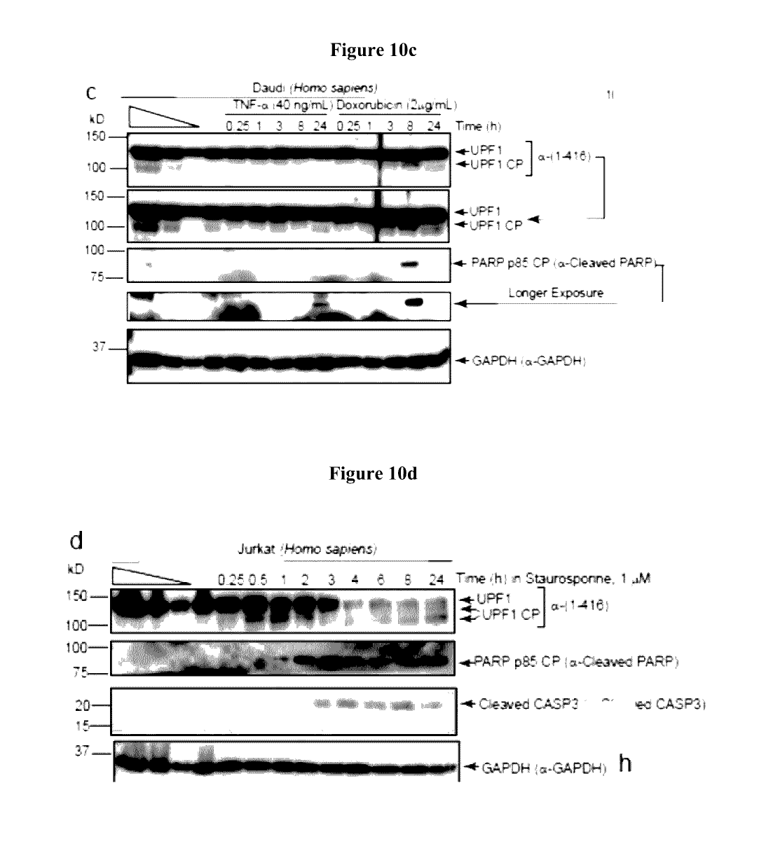

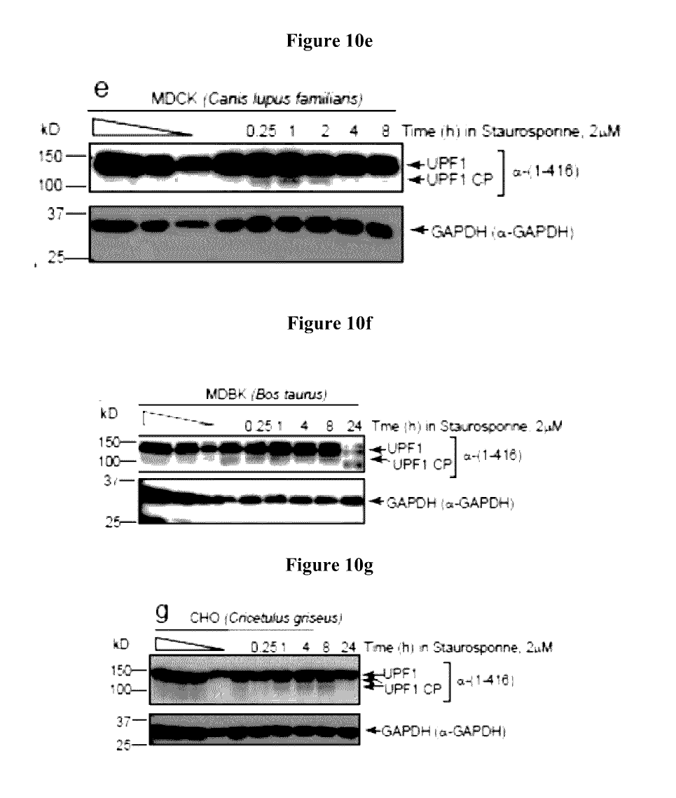

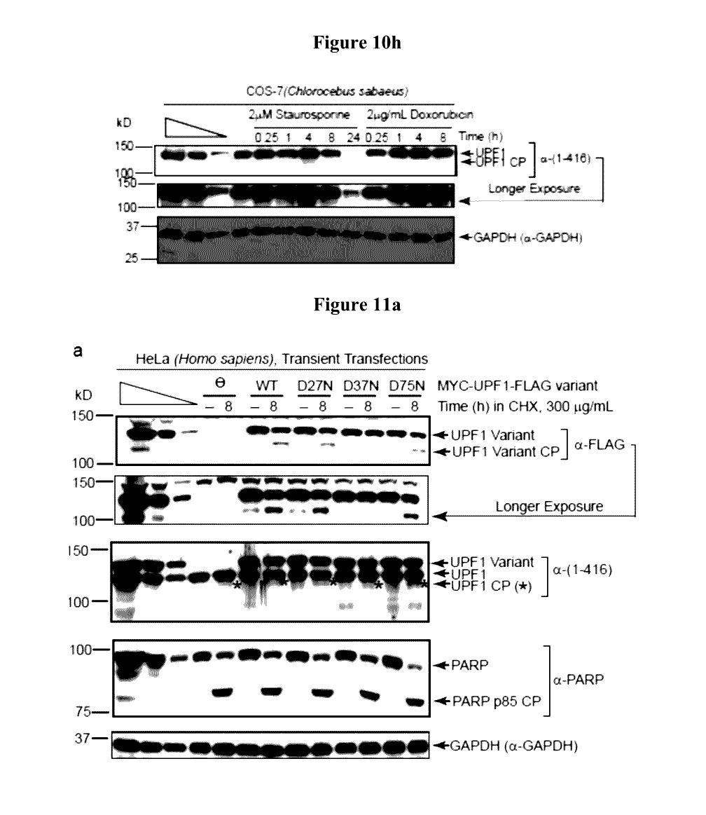

To examine the timing of UPF1 CP generation, HeLa cells were treated with the clinically used topoisomerase inhibitor etoposide (ETP). ETP induced generation of a UPF1 CP before full induction of cleaved initiator caspase 9 (CASP9) and cleaved executioner CASP3 (FIG. 2b). Generation of the UPF1 CP prior to full CASP9 and CASP3 cleavage is recapitulated in human embryonic kidney (HEK)293T cells during CHX treatment (FIG. 10a). The effects of other apoptotic inducers on UPF1 CP generation were examined. Treatment of HEK293T cells with staurosporine also yielded two UPF1 CPs, prior to full cleavage of CASP9 and CASP3 (FIG. 10b). Exposure of the human Daudi B-lymphoblast cell line to either tumor necrosis factor-α (TNF-α) or doxorubicin led to generation of the UPF1 CP prior to maximal PARP cleavage (FIG. 10c). Staurosporine-challenged Jurkat T-cells also yielded a UPF1 CP prior to maximal cleavage of CASP3 or PARP (FIG. 10d).

To probe whether generation of the UPF1 CP is evolutionarily conserved, mouse C2C12 myoblasts were exposed to CHX or ETP, both of which generated a UPF1 CP prior to maximal CASP3 cleavage (FIG. 2c). Exposure of canine (MDCK), bovine (MDBK), and Chinese hamster (CHO) cells to staurosporine led to UPF1 CP production (FIG. 10e-g). Likewise, exposure of African Green monkey (COS-7) cells to staurosporine or doxorubicin yielded a UPF1 CP (FIG. 10h). UPF1 CP levels varied drastically across cell lines, likely for three reasons: (i) in non-human cells, how efficiently these treatments elicited apoptosis cannot be assessed because antibodies to human PARP, cleaved human CASP9, and cleaved human CASP3 do not cross-react; (ii) anti-UPF1 antiserum was raised against the first 416 amino acids of human UPF1 and may exhibit reduced cross-reactivity to non-human UPF1 CP; and (iii) as a result of cleavage at the N-terminus (see below), the human UPF1 CP exhibits less than one-third the immunoreactivity of full-length human UPF1 with this UPF1 antiserum. Notwithstanding this, UPF1 CP generation is an early event that is evolutionarily conserved, indicating that UPF1 cleavage plays a role in the cellular response to apoptotic induction.

### Mapping UPF1 Hydrolysis

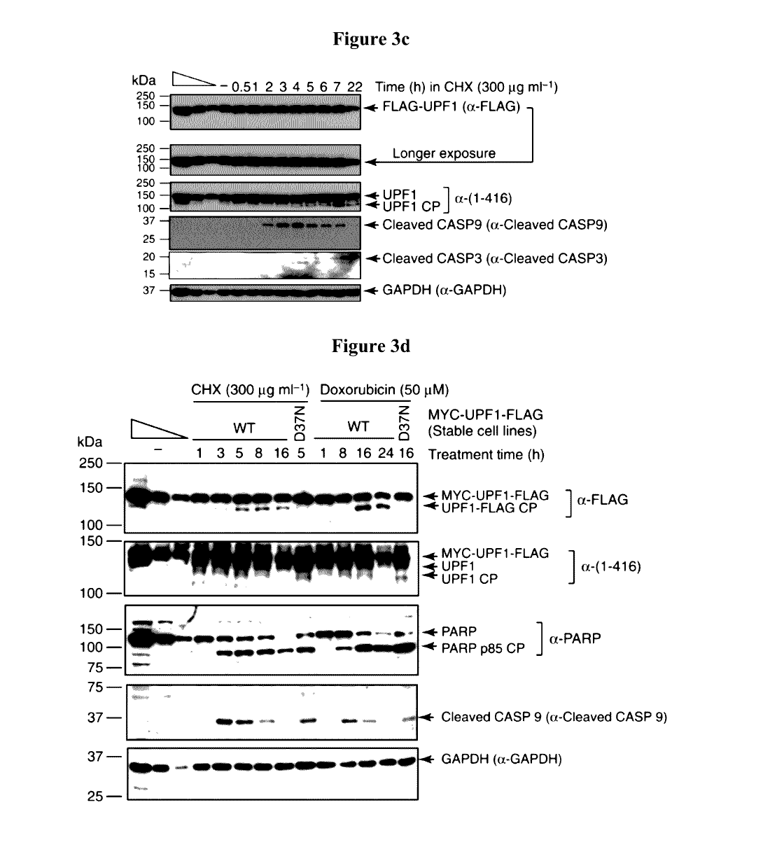

To probe whether caspases are involved in UPF1 CP generation, HEK293T cells were pre-incubated with a panel of caspase inhibitors followed by exposure to CHX (FIG. 3a). Cells treated with each caspase inhibitor showed drastically reduced UPF1 CP levels, with Z-DEVD-fmk and Z-VAD-fmk lowering the level of UPF1 CP to nearly undetectable. Thus, caspases, and/or alternative proteases activated downstream of caspases, are involved in UPF1 CP production. A HeLa cell line stably expressing N-terminally tagged FLAG-UPF1 was exposed to CHX. While anti-FLAG immunoblots failed to reveal any UPF1 CP even after long exposure, anti-UPF1 immunoblots using antiserum raised against amino acids 1-416 (FIG. 3b) yielded detectible UPF1 CP (FIG. 3c), indicating that cleavage occurs within the first 416 amino acids of UPF1 so as to eliminate the FLAG epitope but preserve partial immunoreactivity with the UPF1 anti serum.

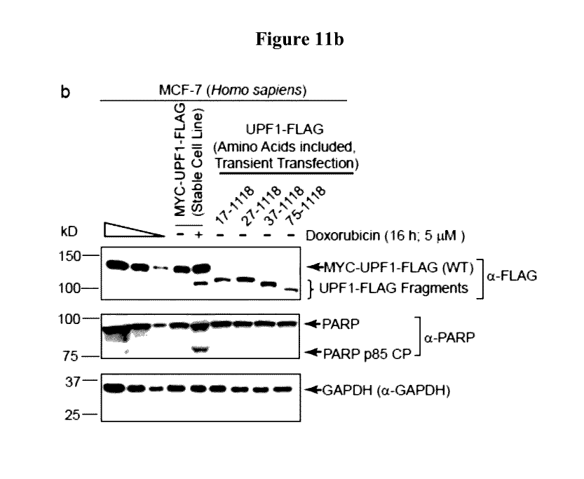

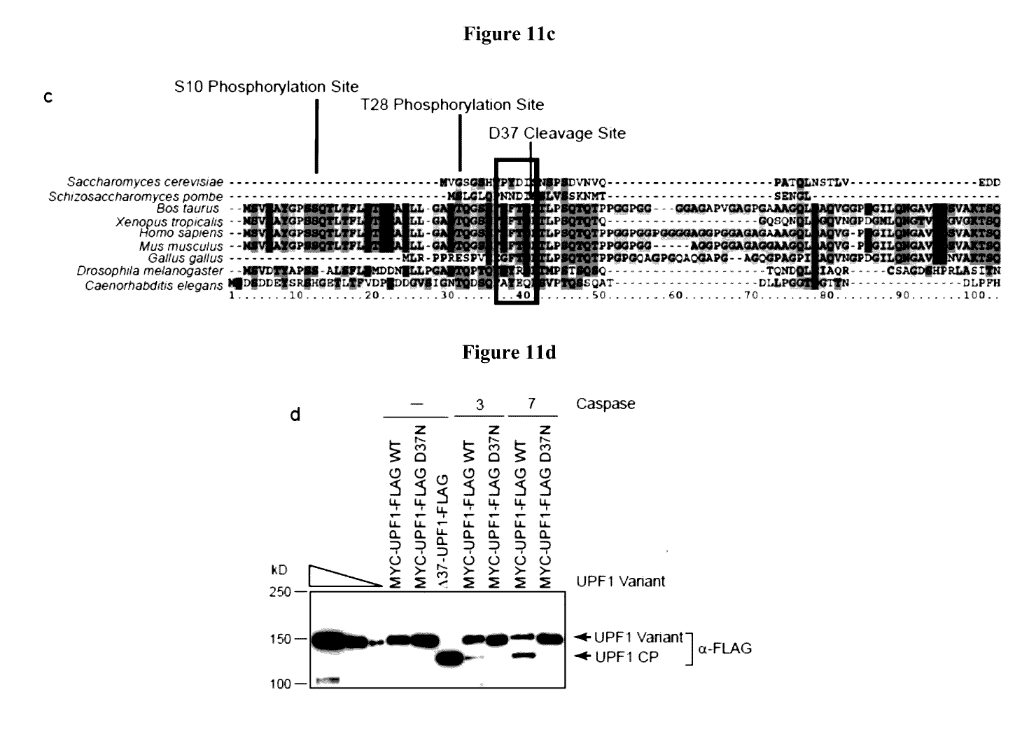

Inventories of in vivo apoptotic cleavage events indicate that cleavage specificity in living cells is determined chiefly by an aspartic acid residue at the P1 position; P4-P2 residues contribute far less to specificity in cells than is indicated by in vitro-derived peptide-based substrate profiles. Accordingly, attention was focused solely on aspartic acid (D) residues in human UPF1 and interrogated residues D27, D37, and D75 near the UPF1 N-terminus by mutating each to asparagine (N). Full-length wild-type (WT) UPF1 and, separately, each variant was expressed bearing an N-terminal MYC-tag and a C-terminal FLAG-tag in HeLa cells at a level equal to endogenous UPF1, and cells were subsequently challenged with CHX. For UPF1 WT, UPF1 D27N and UPF1 D75N, the UPF1 CP was generated at about one-third the level of uncleaved UPF1, as judged using an anti-FLAG immunoblot (FIG. 11a). Both the UPF1 CP and uncleaved UPF1 retained the C-terminal FLAG tag, allowing unambiguous assessment of the ratio of UPF1 CP to full-length UPF1. UPF1 D37N yielded no UPF1 CP, indicating that the amide bond after D37 is the site of hydrolysis (i.e., D37 is the P1 residue).

HeLa cells stably expressing one copy of retrovirally introduced MYC-UPF1-FLAG WT or MYC-UPF1-FLAG D37N transgene were generated. Each protein was expressed at −2.7 fold the level of endogenous UPF1 (FIG. 3d). In these cell lines, the D37N mutation abolished UPF1 CP generation in response to CHX and doxorubicin (FIG. 3d). MCF7 cells stably transduced with MYC-UPF1-FLAG WT also generated the UPF1 CP at about one-third the level of uncleaved UPF1 in response to doxorubicin, and the UPF1 CP matched the molecular weight of a UPF1 fragment encompassing residues 38-1118 (FIG. 11b). The N-terminal 37 amino acid fragment released upon cleavage could not be detected, likely either for technical reasons or because this fragment is unstable. Having established that one cleavage event occurs at the after position 37, the conservation of surrounding amino acids was examined by aligning UPF1 sequences from multiple species using ClustalX (FIG. 11c). The putative consensus cleavage site EFTD is completely conserved in human, bovine, mouse and Xenopus laevis UPF1—it deviates in chicken UPF1 at a single amino acid (where D is G)—and harbors T at the P2 residue, consistent with the high frequency of S and T residues at P4, P3, and P2 residues in cellular apoptotic protein cleavage sites. Previously confirmed caspase substrates also bear similar cleavage sites: protein kinase C ζ is cleaved after EETD, and the NF-kB p65/RelA subunit is cleaved after VFTD.

Which caspase(s) are sufficient to cleave UPF1 in vitro by treating immunoprecipitated samples of full-length MYC-UPF1-FLAG WT or the non-cleavable MYC-UPF1-FLAG D37N variant with recombinant caspases (FIG. 11d) was determined. CASP3 and CASP7 cleaved MYC-UPF1-FLAG WT but not MYC-UPF1-FLAG D37N into a fragment with the same molecular weight as a Δ37-UPF1-FLAG variant lacking the N-terminal MYC-tag and first 37 residues of MYC-UPF1-FLAG WT (recapitulating the mapped UPF1 CP). This is consistent with the observation that Z-DEVD-fmk and Z-VAD-fmk blunt UPF1 CP production (FIG. 3a).

### UPF1 CP is not Functional in NMD

Both serine 10 (S10) and threonine 28 (T28) are phosphorylated by the NMD-associated kinase SMG1, and phosphorylation is critical for NMD. Cleavage at D37 in human UPF1 would cause a loss of these phosphorylation sites and, indeed, experimental truncation of the first 35 amino acids in Arabadopsis thaliana UPF1 (causing loss of three phosphorylation sites) eliminated its NMD activity and causes it to act dominant negatively. A previously described deletion of the N-terminal 63 amino acids of human UPF1 (dNT) causes loss of NMD activity and dominant negative behavior, as does mutation of the threonine 28 phosphorylation site to alanine.

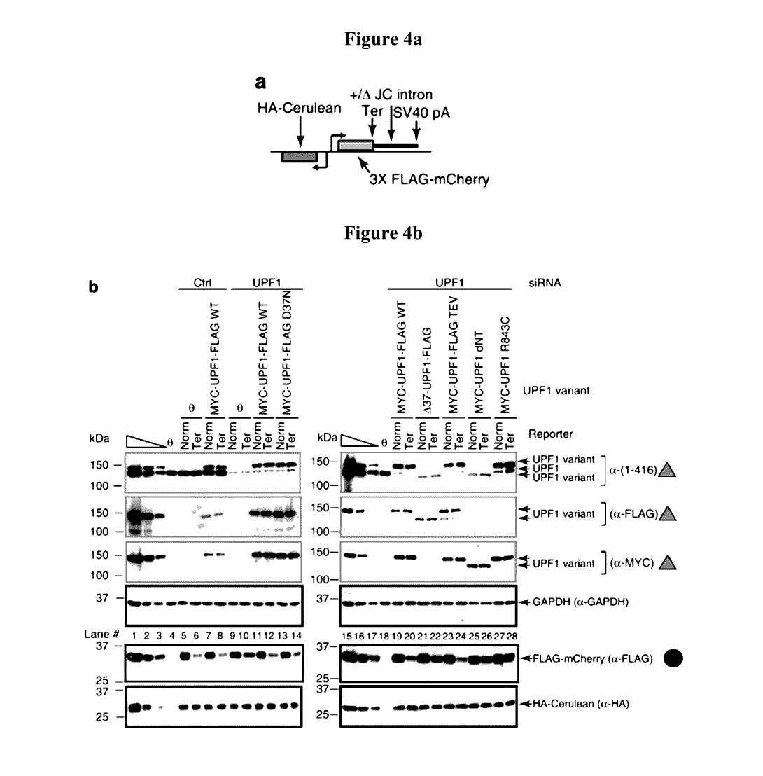

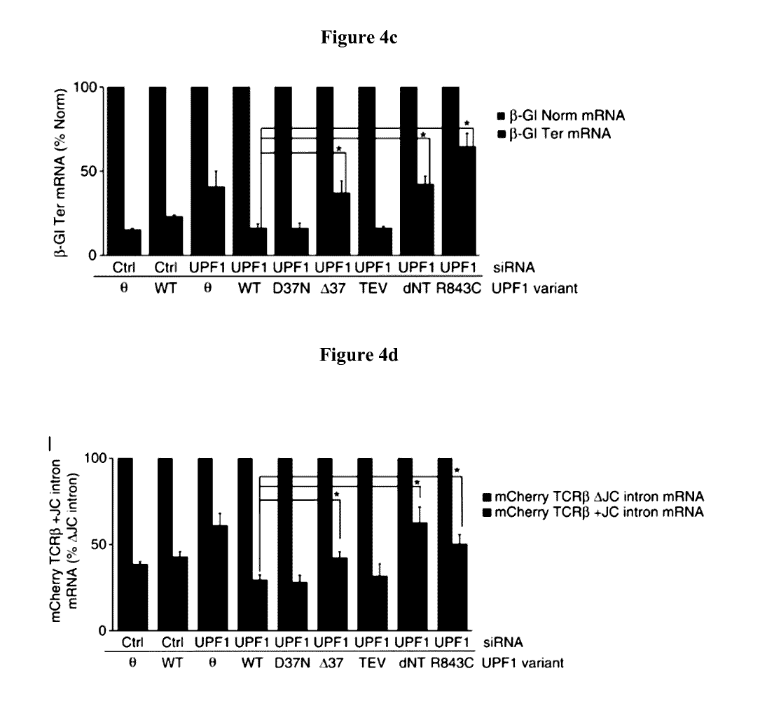

The NMD activity of exogenously expressed UPF1 proteins without endogenous UPF1 was assayed. Endogenous UPF1 levels in HEK293T cells were depleted to <10% of normal using siRNA and one of several siRNA-resistant UPF1 expression vectors: MYC-UPF1-FLAG WT; MYC-UPF1-FLAG D37N; Δ37-UPF1-FLAG; MYC-UPF1-FLAG TEV; MYC-UPF1 dNT; or MYC-UPF1 R843C, which abolishes UPF1 helicase activity http://www.ncbi.nlm.nih.gov/pmc/articles/PMC4375787/−R50 were subsequently transiently introduced. Transfections included either a “Norm” or a “Ter” plasmid set to assess NMD activity. The “Norm” set consists of the β-Gl Norm reporter plasmid, the MUP reference plasmid, and a T-cell receptor (TCR)β-based reporter plasmid. This TCRβ-based reporter plasmid contains a bidirectional promoter driving synthesis of an HA-Cerulean fluorescent protein and, in the opposite orientation, a 3× FLAG-mCherry fluorescent protein whose transcript contains a 3′ UTR composed of a TCRβ minigene lacking introns (Δ JC intron) (FIG. 4a). The “Ter” plasmid set contains the β-Gl Ter reporter plasmid, the MUP reference plasmid, and a TCRβ reporter plasmid bearing (+) the JC intron >55 nt downstream of the mCherry termination codon rendering the mCherry transcript an EJC-mediated NMD substrate (FIG. 4a). Each variant was expressed at a level equivalent to endogenous UPF1 as assessed by comparing anti-UPF1, anti-MYC, and anti-FLAG immunoblots (FIG. 4b).

Comparing the levels of Δ37-UPF1-FLAG and MYC-UPF1 dNT to the level of MYC-UPF1-FLAG WT in immunoblots using the UPF1 aa 1-416 antiserum revealed a >3-fold loss in immunoreactivity despite expression at equivalent levels (as assessed using anti-FLAG and anti-MYC immunoblots; FIG. 4b). Comparing the level of β-Gl Ter mRNA to the level of β-Gl Norm mRNA revealed that Δ37-UPF1-FLAG is unable to promote NMD whereas MYC-UPF1-FLAG WT, MYC-UPF1-FLAG D37N and MYC-UPF1-FLAG TEV can: the β-Gl Ter mRNA level was ˜2.4 fold higher in Δ37-UPF1-FLAG transfectants than in MYC-UPF1-FLAG WT transfectants (FIG. 4c). MYC-UPF1 dNT and MYC-UPF1 R843C were non-functional, yielding β-Gl Ter mRNA levels ˜2.6 and ˜4-fold higher than in MYC-UPF1-FLAG WT transfectants. These results were confirmed with the mCherry-TCR3 reporters (FIG. 4d). The anti-FLAG immunoblot of 3× FLAG-mCherry protein (normalized to HA-Cerulean protein, whose level is unaffected by changes in NMD) revealed that the NMD substrate produces ˜2-fold more protein in cells expressing non-functional Δ37-UPF1-FLAG, MYC-UPF1 dNT, or MYC-UPF1 R843C relative to cells expressing MYC-UPF1-FLAG WT (FIG. 4b). Thus, UPF1 CP fails to support NMD.

### UPF1 CP is a Dominant-Interfering Protein

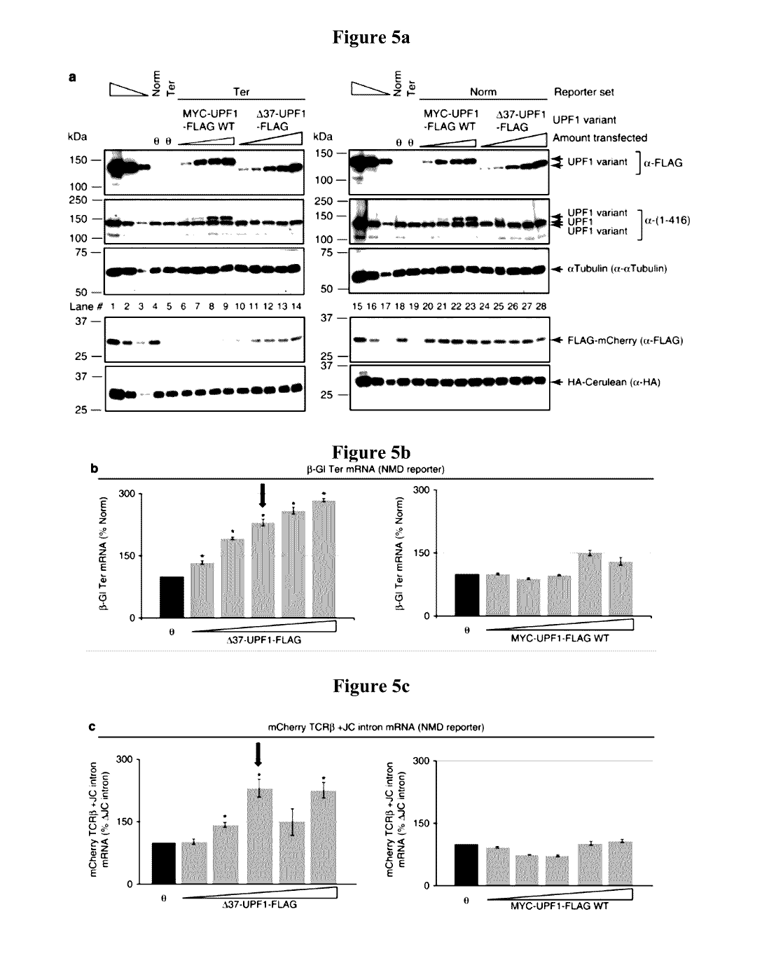

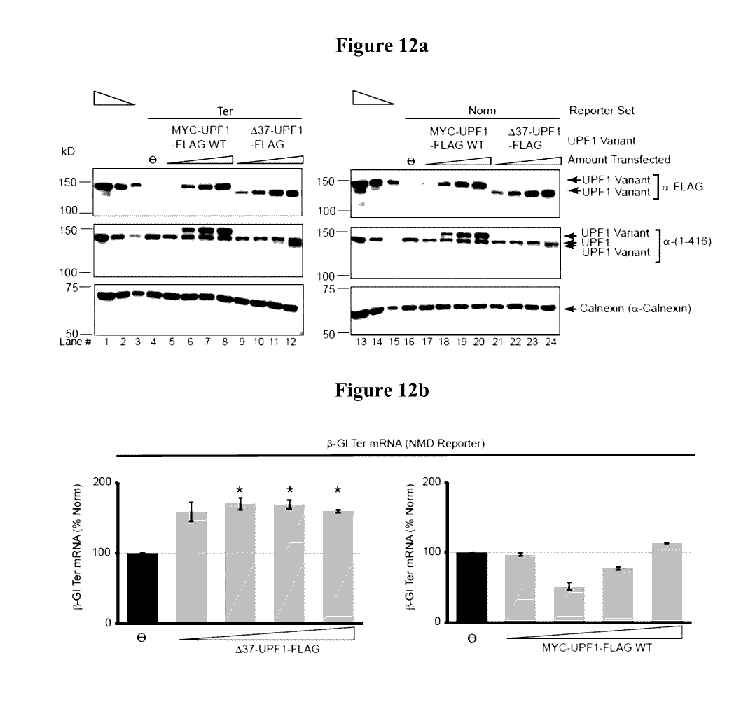

To determine whether UPF1 CP plays a dominant-interfering role in suppressing NMD even at substoichiometric levels relative to uncleaved UPF1, HeLa-cell UPF1 function was challenged by introducing increasing amounts of plasmid DNA to express increasing but sub-stoichiometric amounts of Δ37-UPF1-FLAG or, as a control, MYC-UPF1-FLAG WT; in parallel, empty vector DNA (Θ) was introduced as an additional control (FIG. 5a). These transfections included the “Norm” or “Ter” plasmid sets. While the flexible linker and FLAG epitope of Δ37-UPF1-FLAG limit its complete resolution from endogenous UPF1 (FIG. 5a), the level of Δ37-UPF1-FLAG can be compared to the level of MYC-UPF1-FLAG WT in anti-FLAG blots, and since MYC-UPF1-FLAG WT is cleanly resolved from endogenous UPF1 in anti-UPF1(1-416) blots, it is possible to determine the levels of Δ37-UPF1-FLAG relative to endogenous UPF1 (FIG. 5a). Levels of the β-Gl Ter NMD substrate revealed that, relative to transfections employing empty vector, increasing amounts of the Δ37-UPF1-FLAG elicited an increase in the level of β-Gl Ter mRNA (FIG. 5b). Δ37-UPF1-FLAG expression at about one-third the level of endogenous UPF1 (FIG. 5a) yielded a ˜2.4 fold higher β-Gl Ter mRNA level than in transfections employing empty vector (FIG. 5b, arrow). Δ37-UPF1-FLAG expression increased the level of the NMD substrate mCherry-TCR3+JC intron mRNA ˜2.3 fold (FIG. 5c; in the fourth sample, the decrease to ˜1.5 fold and large error bars are likely due to experimental noise since levels of β-Gl Ter mRNA continue to increase with increasing Δ37-UPF1-FLAG levels). The amount of 3×FLAG-mCherry that derived from mCHERRY-TCRβ+JC intron mRNA increased with increasing yet substoichiometric amounts of Δ37-UPF1-FLAG (FIG. 5a). Almost no changes were observed in the level of mCherry-TCR3+JC intron mRNA or its product protein when endogenous UPF1 was challenged with increasing but substoichiometric levels of MYC-UPF1-FLAG WT (FIG. 5a,c). It was confirmed that challenge of endogenous UPF1 in HEK293T cells with increasing amounts of Δ37-UPF1-FLAG, relative to empty vector control, increased β-Gl Ter mRNA levels, whereas MYC-UPF1-FLAG WT had no such effect (FIG. 12a,b).

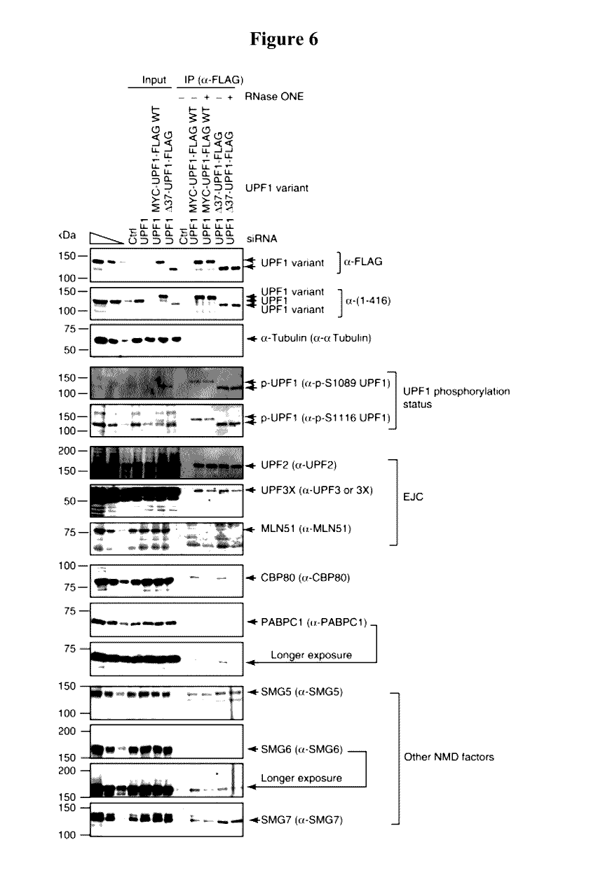

An attempt to rule out the explanation for the lack of Δ37-UPF1-FLAG function in NMD, i.e., that the truncated protein is misfolded, by characterizing the composition of the RNP containing either MYC-UPF1-FLAG WT or Δ37-UPF1-FLAG, was made. HEK293T cells were depleted of endogenous UPF1 using siRNA, and either MYC-UPF1-FLAG WT or Δ37-UPF1-FLAG was expressed at a level equivalent to the normal level of endogenous UPF1 (FIG. 6). MYC-UPF1-FLAG WT or Δ37-UPF1-FLAG complexes were immunoprecipitated from lysates using anti-FLAG resin, each immunoprecipitate was divided in half, and one half was incubated with BSA while the other half was incubated with RNase ONE to identify protein-protein interactions that are stabilized by RNA.

Immunoblotting using antibodies directed against p-S1089 or p-S1116 in UPF1 revealed slightly enhanced phosphorylation of Δ37-UPF1-FLAG relative to MYC-UPF1-FLAG WT (FIG. 6). Accumulation of C-terminal phosphates is a feature of ATPase-deficient UPF1 variants that cannot support NMD. Equivalent levels of EJC components UPF2, UPF3× and MLN51, the cap-binding protein CBP80, and the poly(A) binding protein (PABP)C1 were co-immunoprecipitated with both UPF1 variants (FIG. 6). Δ37-UPF1-FLAG retrieved slightly increased levels of SMG5 and SMG7 relative to MYC-UPF1-FLAG WT in RNase-insensitive interactions (FIG. 6). Equivalent levels of SMG6 were retrieved in a partially RNase-sensitive interaction (FIG. 6). SMG6 association with a region outside of the UPF1 N-terminus is consistent with several recent reports. These results indicate that gross misfolding of the UPF1 CP cannot explain its nonfunctional and dominant-interfering behavior.

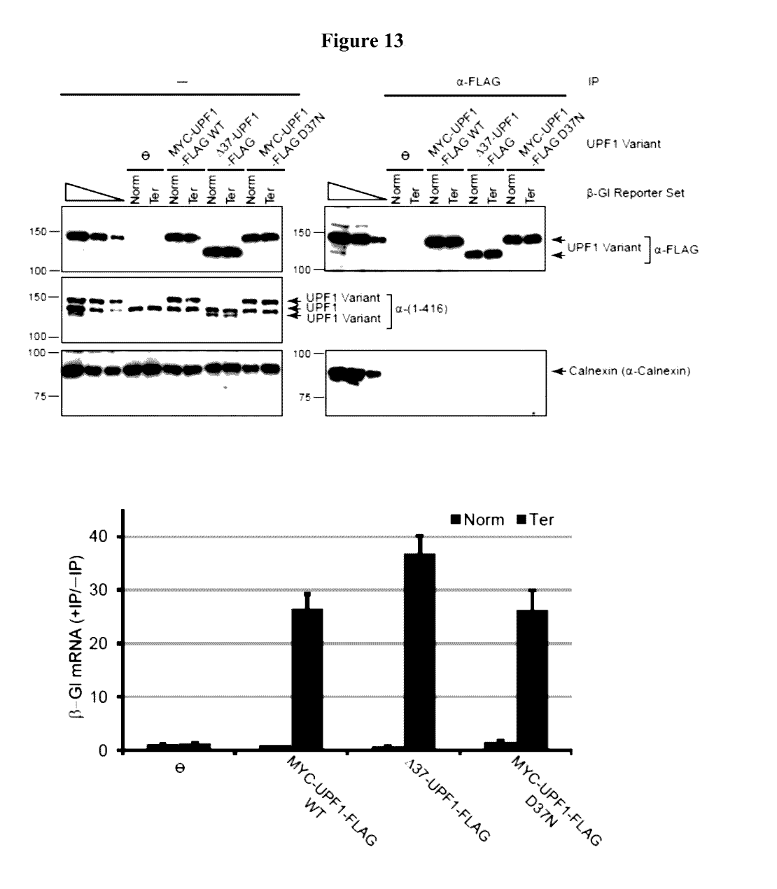

The binding of MYC-UPF1-FLAG WT, Δ37-UPF1-FLAG, and MYC-UPF1-FLAG D37N to PTC-containing mRNAs relative to their PTC-free counterparts by transfecting cells expressing equivalent levels of each UPF1 variant with a combination of plasmids encoding β-Gl Ter mRNA and MUP mRNA, or separately, plasmids encoding the β-Gl Norm mRNA and MUP mRNA was characterized. The binding of each variant to β-Gl Ter mRNA and to its PTC-free counterpart (FIG. 13) in immunoprecipitates was measured. As previously reported, MYC-UPF1-FLAG WT retrieved ˜26-fold higher levels of the PTC-containing mRNA when adjusted for expression levels and MYC-UPF1-FLAG D37N did likewise. Like the nonfunctional dNT UPF1 variant as well as a non-functional 4SA variant lacking four phosphorylation sites, Δ37-UPF1-FLAG also retrieved β-Gl Ter mRNA relative to β-Gl Norm mRNA with an efficiency that was comparable to that of MYC-UPF1-FLAG WT and MYC-UPF1-FLAG D37N (˜36-fold enrichment), despite being non-functional (FIGS. 4, 5). In conclusion, like the dNT variant, Δ37-UPF1-FLAG is not misfolded—it can bind to the same complement of proteins as wild-type UPF1 and is enriched on a PTC-bearing transcript. Rather, a defect in the NMD cycle after RNA binding occurs.

### UPF1 Cleavage Upregulates Genes Involved in Apoptosis

91 genes upregulated upon UPF1 downregulation in Mendell et al. (“Nonsense surveillance regulates expression of diverse classes of mammalian transcripts and mutes genomic noise,” Nat Genet. 2004; 36:1073-8) were interrogated using the online DAVID gene ontology tool to cluster genes by function in order to determine the physiological relevance of UPF1 cleavage and the attenuation of NMD for cells exposed to chemotherapeutics that cause apoptosis. A cluster (11 genes) was found under “positive regulation of programmed cell death” (p=2.3E-4) as well as a group of genes belonging to “p53 signaling pathway” (4 genes) and “regulation of cell cycle” (5 genes). Results from Viegas et al. (“The abundance of RNPS1, a protein component of the exon junction complex, can determine the variability in efficiency of the Nonsense Mediated Decay pathway,” Nucleic Acids Res. 2007; 35:4542-51) showed a cluster (20 genes) under “positive regulation of programmed cell death” (p=7.4E-8). DAVID analysis of results from Cho et al. (“Staufenl-mediated mRNA decay functions in adipogenesis,” Mol Cell. 2012; 46:495-506) also yielded genes in “positive regulation of programmed cell death” (10 genes) and regulation of cell cycle (5 genes). Indeed, NMD targets that were previously analyzed (FIG. 9a) include CDKN1A mRNA, which encodes the classical cell-cycle inhibitory protein p21, and GADD45α and GADD45β mRNAs, which produce proteins involved in cell-cycle arrest that also transiently upregulate CASP3 and CASP7 to promote apoptosis. Thus, it was hypothesized that, among the transcripts upregulated upon NMD attenuation are a group that the cell can exploit in response to apoptotic inducers.

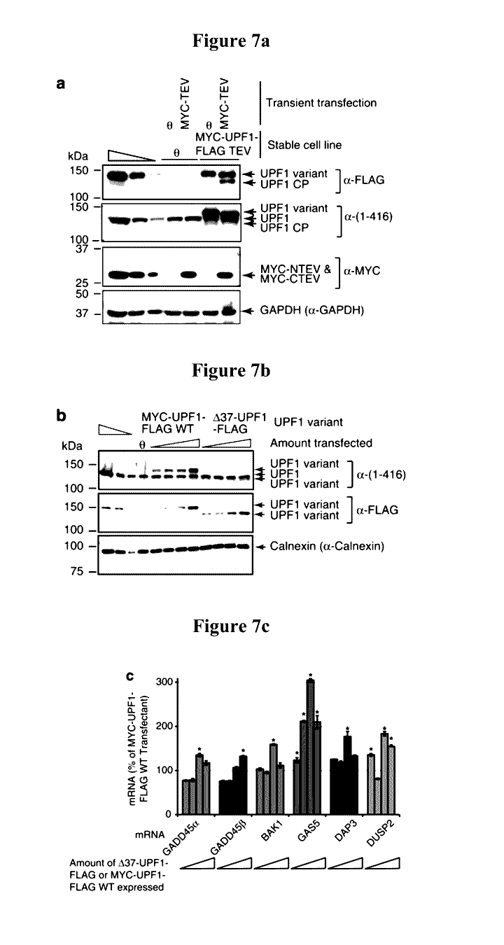

Stably transduced HeLa-cell lines bearing either empty vector or a fully functional MYC-UPF1-FLAG TEV allele that harbors the tobacco etch virus (TEV) protease cleavage site substituted into the D37 position (FIG. 4) were generated. Since TEV protease has no cleavage sites in the mammalian proteome, transfection with a plasmid encoding two complementary MYC-tagged TEV protease fragments expressed from a bidirectional promoter allows MYC-UPF1-FLAG TEV to be specifically cleaved in living cells in the absence of apoptotic inducers (FIG. 7a). To identify changes in cellular mRNAs that are due to UPF1 CP production, RNA-Seq was performed on the empty-vector cell line and the MYC-UPF1-FLAG TEV cell line, both in the presence or absence of TEV expression, with the assumption that direct and indirect NMD targets could be uncovered.

Abundance changes in either class of targets may have important effects on cellular physiology. To control for differences in the cellular responses to plasmid identity and transfection, changes in mRNA abundance of the MYC-UPF1-FLAG TEV cell line with and without TEV protease were normalized to changes in the empty vector cell line with and without TEV protease. Upregulation of mRNAs was recovered for CDKN1A (˜3-fold), GADD45α (˜3.7-fold) and GADD45β (4.7-fold). By expressing increasing amounts of MYC-UPF1-FLAG WT or Δ37-UPF1-FLAG in either HeLa (FIG. 7b) or MCF7 (FIG. 7d) cells and measuring the resultant changes in mRNA abundance (FIG. 7c, FIG. 7e), the upregulation of a subset of additional genes (GADD45α, GADD45β, BAK1, GAS5, DAP3, DUSP2 and BCL3), each of which has individual literature-documented roles in promoting cell-cycle arrest or apoptosis when its expression is increased was verified. While the decrease observed for most mRNA ratios at the highest level of Δ37-UPF1-FLAG in HeLa cells cannot be explained (but not in MCF7 cells), it is noted that this expression level is greater than that observed for the CP in doxorubicin-treated cells.

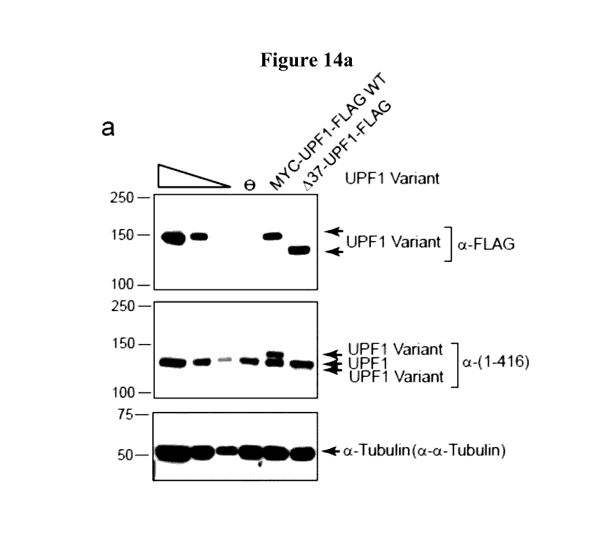

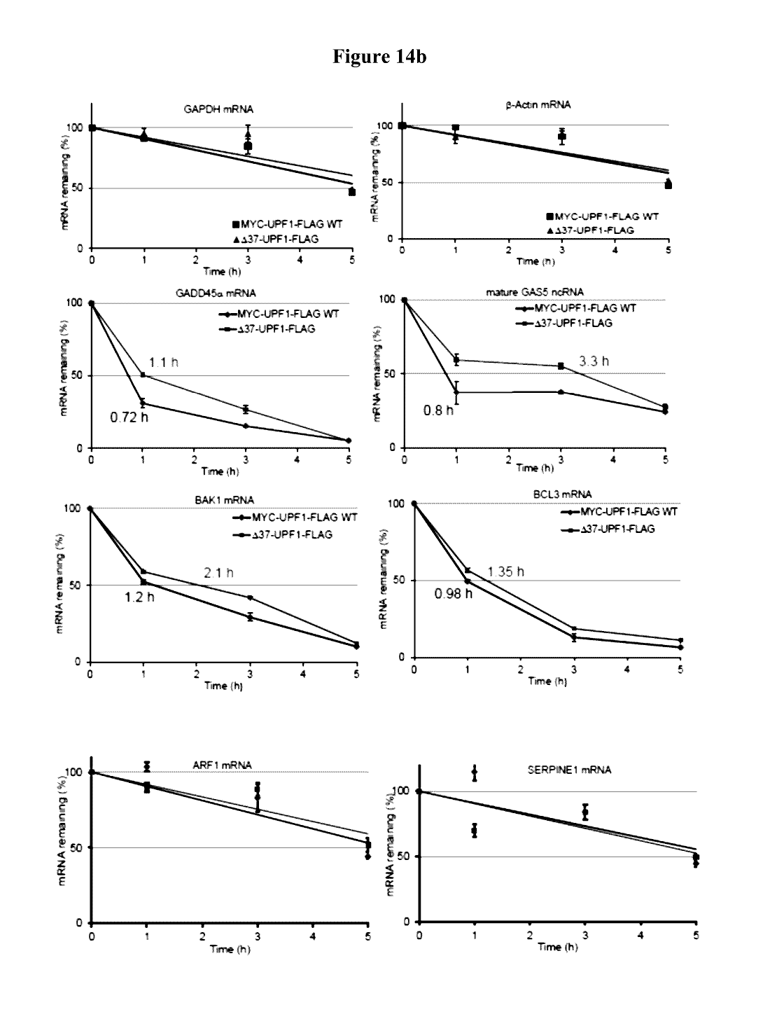

To support these observations, HeLa cells were transfected with substoichiometric amounts of either MYC-UPF1-FLAG WT or an equivalent amount of Δ37-UPF1-FLAG (FIG. 14), cells were treated 48 h later with 5,6-dichloro-1-β-D-ribofuranosyl-1H-benzimidazole (DRB), and the half-lives of endogenous NMD targets after DRB-mediated transcriptional arrest were analyzed. In contrast to GAPDH and β-actin mRNAs, there were noted increases in the stability of GAS5 ncRNA as well as GADD45α, BAK1, and BCL3 mRNAs. Stability of ARF1 and SERPINE1 mRNAs (two SMD targets) were unaffected (FIG. 14). Thus, UPF1 CP can partially attenuate NMD levels at substoichiometric amounts. Each of the genes verified from the RNA-seq data can individually promote either cell cycle arrest or apoptosis and thus could be exploited by cells in response to chemotherapeutic treatment.

### Modulation of NMD Activity Affects Doxorubicin Sensitivity

Two testable hypotheses follow from the observation that generating UPF1 CP in the absence of chemotherapy augments the expression of genes involved in apoptotic progression. First, inhibiting UPF1 CP production should slow the cell-death response to doxorubicin. Second, inhibiting NMD through exogenous introduction of UPF1 CP or small-molecule treatment should promote doxorubicin-mediated cell death.

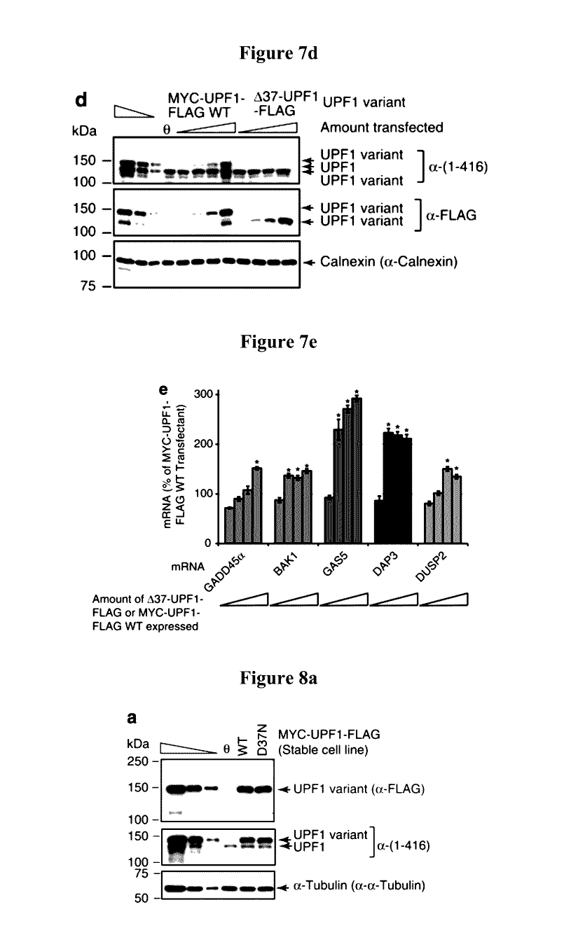

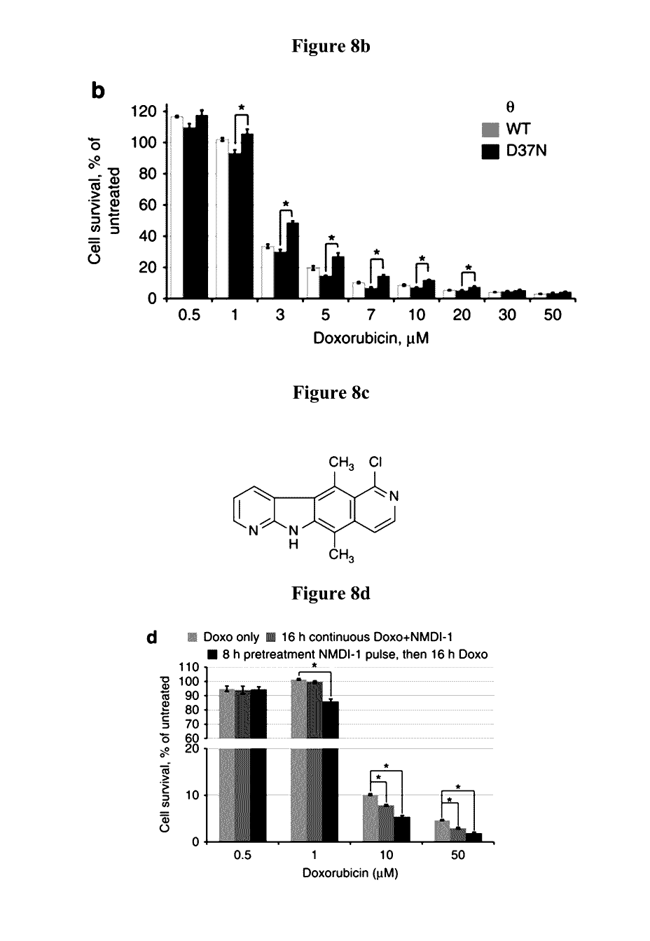

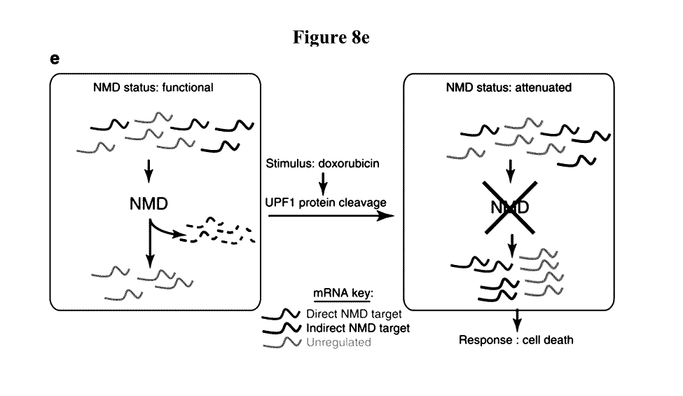

To test the first hypothesis, HeLa cell lines stably expressing MYC-UPF1-FLAG WT or non-cleavable MYC-UPF1-FLAG D37N (FIGS. 3d, 4b, 8a), both of which support NMD, were utilized. Each was expressed at ˜2.7 fold above the level of endogenous UPF1 and, more importantly, at levels identical to one another (FIG. 8a). These cell lines were exposed to a range of doxorubicin concentrations and cell viability was assessed after 16 h using an assay that detects ATP generation by living cells. At a sub-lethal doxorubicin concentration (0.5 μM), no statistically significant difference in viability was detected. However, as doxorubicin toxicity increased, the MYC-UPF1-FLAG D37N cell line showed increased resistance to death relative to the MYC-UPF1-FLAG WT cell line, reaching a maximum of −2.2-fold greater survival.

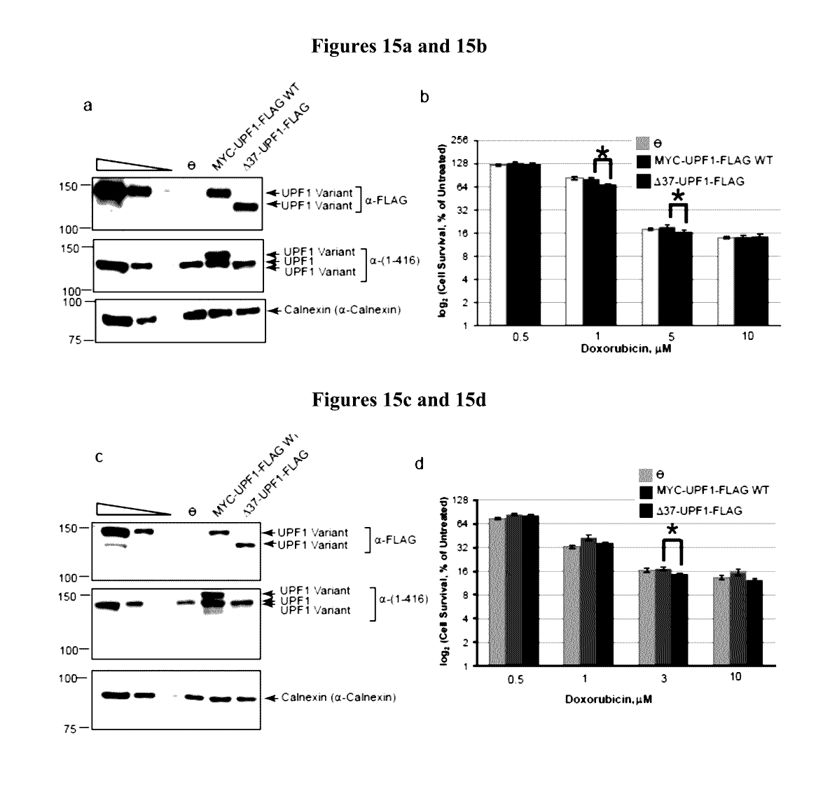

With the first hypothesis verified, either MYC-UPF1-FLAG WT or Δ37-UPF1-FLAG were transiently expressed in HeLa cells (FIG. 15a) or MCF-7 cells (FIG. 15c) and transfectants were challenged with doxorubicin. Although statistically significant increases in sensitivity were observed for Δ37-UPF1-FLAG transfectants relative to MYC-UPF1-FLAG WT transfectants, the effect was mild in both cell types (FIG. 15b, d), likely because the toxic effects of lipofection obscure differences between the two transfected cell populations and limit the dynamic range of the assay.

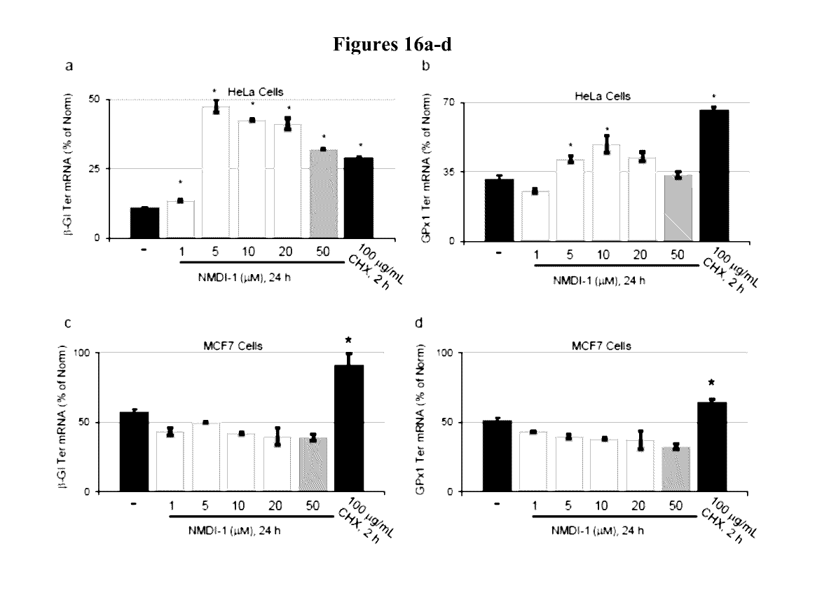

Therefore, a small-molecule inhibitor of NMD, NMDI-1 (FIG. 8c), that interferes with the interaction between UPF1 and SMG5 was utilized. Application of NMDI-1 and the consequential attenuation of NMD may more fully replicate the complete inhibition of NMD mediated by the UPF1 CP generation explored here as well as UPF1 dephosphorylation and the generation of additional UPF1 CPs seen with doxorubicin (FIG. 1c). NMDI-1 is effective in HeLa cells, raising the levels of β-Gl Ter mRNA and another NMD target—a PTC-bearing glutathione peroxidase 1(GPx1) mRNA-˜3.8-fold and ˜1.6 fold, respectively, at a concentration of 10 which was used in subsequent experiments (FIG. 16a,b).

Since NMDI-1 had no effect in MCF7 cells (FIG. 16c, d), HeLa cells were focused on. Because of results indicating that the efficacy of combination small-molecule treatments is affected by both drug order and timing, three treatment regimens were considered. First, HeLa cells were challenged with various concentrations of doxorubicin alone. Second, cells were continuously co-incubated for 16 h with doxorubicin and NMDI-1. Third, a transient pulse of NMDI-1 was applied for 8 h, cells were washed to remove NMDI-1, and doxorubicin was then applied. At sub-lethal doses of doxorubicin (0.5 μM), none of the treatments significantly affected viability (FIG. 8d). Confirming the hypothesis that inhibiting NMD should increase sensitivity to doxorubicin, continuous co-treatment with NMDI-1 led to statistically significant decreases in cell viability relative to doxorubicin treatment alone (FIG. 8d). Transient pre-treatment with NMDI-1 led to an even more pronounced effect and up to a ˜2.5 fold reduction in cell viability at 50 μM doxorubicin relative to doxorubicin alone (FIG. 8d), despite the total time of exposure to NMDI-1 being half of that in the co-treatment regimen. Thus, inhibiting NMD promotes doxorubicin-mediated cell death, and conversely, inhibiting UPF1 CP generation obscures this effect.

These studies show that NMD activity is blunted during chemotherapeutic treatments (doxorubicin, staurosporine, etc.) that ultimately cause apoptosis. During treatment with doxorubicin and other clinically relevant small molecules (e.g., etoposide), one or more UPF1 CPs are produced. The UPF1 CP was mapped to a region encompassing UPF1 amino acids 38-1118 that acts to inhibit NMD in dominant-interfering fashion, i.e., at substoichiometric levels relative to cellular UPF1 (FIG. 5; FIG. 12). Inhibition is tunable—the more UPF1 CP is generated, the more PTC-containing reporter mRNA is stabilized (FIG. 5b,c). Increases in PTC-reporter mRNAs (˜2.4-fold; FIG. 5b) are less than those achievable using UPF1 ablation (˜6-10-fold), and fold-changes in endogenous NMD targets are smaller, indicating that substoichiometric generation of UPF1 CP may be a way to fine-tune gene expression with physiological consequences (FIG. 8). The combined effects of this UPF1 CP, as well as additional UPF1 CPs (FIG. 1c,d), and changes to UPF1 phosphorylation status (FIG. 1c) all likely contribute to inhibition of NMD and upregulation of cell-cycle inhibitory and apoptosis-promoting transcripts seen during doxorubicin treatment (FIG. 1a; FIG. 7). Small molecule-mediated inhibition of NMD could provide an improved therapeutic strategy when delivered in combination with cytotoxic agents already in clinical use (FIG. 8d).

Transient pre-treatment with NMDI-1 before addition of doxorubicin leads to enhanced cell death relative to either doxorubicin alone or to continuous co-treatment of NMDI-1 and doxorubicin. This provides a model for NMD involvement in enabling the establishment of different cellular states by sculpting the mRNA milieu (FIG. 8e). Transcription produces mRNAs that are or are not NMD targets, or are indirect NMD targets. NMD activity degrades NMD-sensitive transcripts that are either not allowed into the pool of translated mRNAs or allowed at only low levels. Shown herein is that NMD activity is under the purview of the cell: NMD activity is tuned by generating a UPF1 CP (FIG. 5) that inhibits NMD, allows increased amounts of NMD-sensitive transcripts into the mRNA milieu, and also regulates indirect NMD targets.

Which transcripts are direct NMD targets or indirect NMD targets may be an academic distinction to the cell. Clearly, inhibiting NMD is able to change the mRNA milieu to promote physiological consequences (FIG. 8). The sum effect of these changes to the mRNA pool alters the cellular state to one that is competent to respond (via death) to the insult that elicited the inhibition of NMD (doxorubicin). Such a model explains why transient pre-treatment with NMDI-1 before application of doxorubicin is a more effective treatment regimen than mere co-treatment of NMDI-1 and doxorubicin. During the pre-treatment pulse, the cell has already attenuated NMD and established an mRNA milieu that can respond to doxorubicin even before doxorubicin is applied, making the response to doxorubicin (death) more rapid. An important caveat to this model is that NMD inhibition also increases the levels of truncated aberrant proteins that have detrimental effects on cellular metabolism. However, the overall response is the same, namely, increased sensitivity to doxorubicin.

In summary, NMD limits the production of aberrant mRNAs containing a premature termination codon and also controls the levels of endogenous transcripts. As shown herein, when human cells are treated used chemotherapeutic compounds, NMD activity declines partly as a result of the proteolytic production of a dominant-interfering form of the key NMD factor UPF1. Production of cleaved UPF1 functions to upregulate genes involved in the response to apoptotic stresses. The biological consequence is the promotion of cell death. Combined exposure of cells to a small molecule inhibitor of NMD, NMDI-1, and the chemotherapeutic doxorubicin leads to enhanced cell death, while inhibiting UPF1 cleavage protects cells from doxorubicin challenge.

What is claimed is:

1. A method of treating cancer in a subject comprising administering to the subject with cancer a therapeutically effective amount of an agent that inhibits nonsense-mediated RNA decay (NMD) and a therapeutically effective amount of a chemotherapeutic agent.

2. The method of claim 1, wherein the agent that inhibits NMD is administered to the subject prior to administration of the chemotherapeutic agent.

3. The method of claim 1, wherein the agent that inhibits NMD is administered to the subject one or more times prior to administration of the chemotherapeutic agent.

4. The method of claim 1, wherein the agent that inhibits NMD is administered to the subject one or more hours prior to administration of the chemotherapeutic agent.

5. The methods of claim 1, wherein the agent that inhibits NMD is administered to the subject one or more days prior to administration of the chemotherapeutic agent.

6. The method of claim 1, wherein the chemotherapeutic agent is doxorubicin.

7. The method of claim 1, wherein the agent that inhibits NMD is NMD1-1.

8. The method of claim 1, wherein the cancer is selected from leukemia, breast cancer, bone cancer, lung cancer and brain cancer.

9. A method of increasing apoptosis in a cancer cell comprising contacting the cell with effective amount of an agent that inhibits nonsense-mediated RNA decay (NMD) and an effective amount of a chemotherapeutic agent.

10. The method of claim 9, wherein the cell is contacted with the agent that inhibits NMD prior to contacting the cell with the chemotherapeutic agent.

11. The method of claim 9, wherein the cell is contacted with the agent that inhibits NMD one or more times prior to contacting the cell with the chemotherapeutic agent.

12. The method of claim 9, wherein the cell is contacted with the agent that inhibits NMD one or more hours prior to contacting the cell with the chemotherapeutic agent.

13. The methods of claim 9, wherein the cell is contacted with the agent that inhibits NMD one or more days prior to contacting the cell with the chemotherapeutic agent.

14. The method of claim 9, wherein the chemotherapeutic agent is doxorubicin.

15. The method of claim 9, wherein the agent that inhibits NMD is NMD1-1.

16. The method of claim 9, wherein the cancer cell is selected from a blood cell, a breast cancer cell, a bone cancer cell, a lung cancer cell and a brain cancer cell.

17. The method of claim 9, wherein in the cell is in vitro or in vivo.

18. A method of increasing the sensitivity of a cancer cell to a chemotherapeutic agent comprising contacting the cell with effective amount of an agent that inhibits nonsense-mediated RNA decay (NMD) and an effective amount of a chemotherapeutic agent.

19. The method of claim 18, wherein the cell is contacted with the agent that inhibits NMD prior to contacting the cell with the chemotherapeutic agent.

20. The method of claim 18, wherein the cell is contacted with the agent that inhibits NMD one or more times prior to contacting the cell with the chemotherapeutic agent.

21. The method of claim 18, wherein the cell is contacted with the agent that inhibits NMD one or more hours prior to contacting the cell with the chemotherapeutic agent.

22. The methods of claim 18, wherein the cell is contacted with the agent that inhibits NMD one or more days prior to contacting the cell with the chemotherapeutic agent.

23. The method of claim 18, wherein the chemotherapeutic agent is doxorubicin.

24. The method of claim 18, wherein the agent that inhibits NMD is NMD1-1.

25. The method of claim 18, wherein the cancer cell is selected from a blood cell, a breast cancer cell, a bone cancer cell, a lung cancer cell and a brain cancer cell.

26. The method of claim 18, wherein in the cell is in vitro or in vivo.


SEQUENCE LISTING (83 sequences)

SEQ ID NO: 1 - DNA, 21 bp, Artificial sequence
  Synthetic construct
gaugcaguuccgcuccauutt

SEQ ID NO: 2 - DNA, 49 bp, Artificial sequence
  Synthetic construct
atatgcagatctgccaccatggagcagaagctgatctcagaggaggacc

SEQ ID NO: 3 - DNA, 74 bp, Artificial sequence
  Synthetic construct
atcgatatcgatttacttatcgtcgtcatccttgtaatcgccacctgatccgccatactgggacagccccgtca

SEQ ID NO: 4 - DNA, 40 bp, Artificial sequence
  Synthetic construct
gcatgcatagatctgccaccatgacggaggaggccgagct

SEQ ID NO: 5 - DNA, 44 bp, Artificial sequence
  Synthetic construct
gcatgcatagatctgccaccatgacacagggctccgagttcgag

SEQ ID NO: 6 - DNA, 50 bp, Artificial sequence
  Synthetic construct
gcatgcatagatctgccaccatgtttactcttcctagccagacgcagacg

SEQ ID NO: 7 - DNA, 38 bp, Artificial sequence
  Synthetic construct
gcatgcatagatctgccaccatggcgcaggttgggccc

SEQ ID NO: 8 - DNA, 49 bp, Artificial sequence; identical to SEQ ID NO: 2
  Synthetic construct

SEQ ID NO: 9 - DNA, 27 bp, Artificial sequence
  Synthetic construct
aggaagagtaaagttggtgaactcgaa

SEQ ID NO: 10 - DNA, 27 bp, Artificial sequence
  Synthetic construct
ttcgagttcaccaactttactcttcct

SEQ ID NO: 11 - DNA, 74 bp, Artificial sequence; identical to SEQ ID NO: 3
  Synthetic construct

SEQ ID NO: 12 - DNA, 49 bp, Artificial sequence; identical to SEQ ID NO: 2
  Synthetic construct

SEQ ID NO: 13 - DNA, 57 bp, Artificial sequence
  Synthetic construct
gctaggaagagtaaaggactggaagtagagattttcggtgaactcgaactcggagcc

SEQ ID NO: 14 - DNA, 48 bp, Artificial sequence
  Synthetic construct
gaaaatctctacttccagtcctttactcttcctagccagacgcagacg

SEQ ID NO: 15 - DNA, 74 bp, Artificial sequence; identical to SEQ ID NO: 3
  Synthetic construct

SEQ ID NO: 16 - DNA, 39 bp, Artificial sequence
  Synthetic construct
gcatcgatcgatgccaccatggccaagttgaccagtgcc

SEQ ID NO: 17 - DNA, 30 bp, Artificial sequence
  Synthetic construct
ctgactggtacctcagtcctgctcctcggc

SEQ ID NO: 18 - DNA, 25 bp, Artificial sequence
  Synthetic construct
cgtctcactagtctcgtgcagatgg

SEQ ID NO: 19 - DNA, 33 bp, Artificial sequence
  Synthetic construct
actgctatcgatcttgggctgcaggtcgaaagg

SEQ ID NO: 20 - DNA, 35 bp, Artificial sequence
  Synthetic construct
gcatgcggtaccccgcgtctggaacaatcaacctc

SEQ ID NO: 21 - DNA, 34 bp, Artificial sequence
  Synthetic construct
actcgactggtacctgaggtgtgactggaaaacc

SEQ ID NO: 22 - DNA, 76 bp, Artificial sequence
  Synthetic construct
atgcatacgcgtgccaccatgtacccatacgatgttccagattacgctggcggaggtatggtgagcaagggcgagg

SEQ ID NO: 23 - DNA, 39 bp, Artificial sequence
  Synthetic construct
atgcatgcggccgctcacttgtacagctcgtccatgccg

SEQ ID NO: 24 - DNA, 60 bp, Artificial sequence
  Synthetic construct
aaagatcatgacatcgattacaaggatgacgatgacaagggcggaggtatggtgagcaag

SEQ ID NO: 25 - DNA, 59 bp, Artificial sequence
  Synthetic construct
cgtataatgtatgctatacgaagttatatcagtcagtcacttgtacagctcgtccagcc

SEQ ID NO: 26 - DNA, 84 bp, Artificial sequence
  Synthetic construct
gcatgcatgggcccgccaccatggactacaaagaccatgacggtgattataaagatcatgacatcgattacaaggatgacgatg

SEQ ID NO: 27 - DNA, 86 bp, Artificial sequence
  Synthetic construct
gtacccatcagggatatctcctttctccgtgctgtcagcgacataacttcgtataatgtatgctatacgaagttatatcagtcagt

SEQ ID NO: 28 - DNA, 29 bp, Artificial sequence
  Synthetic construct
cggagaaaggagatatccctgatgggtac

SEQ ID NO: 29 - DNA, 52 bp, Artificial sequence
  Synthetic construct
cttcgtataatgtatgctatacgaagttattcaacgaggaaggtggtcaggg

SEQ ID NO: 30 - DNA, 29 bp, Artificial sequence
  Synthetic construct
cggagaaaggagatatccctgatgggtac

SEQ ID NO: 31 - DNA, 55 bp, Artificial sequence
  Synthetic construct
gcatgcgggcccataacttcgtataatgtatgctatacgaagttattcaacgagg

SEQ ID NO: 32 - DNA, 84 bp, Artificial sequence; identical to SEQ ID NO: 26
  Synthetic construct

SEQ ID NO: 33 - DNA, 55 bp, Artificial sequence; identical to SEQ ID NO: 31
  Synthetic construct

SEQ ID NO: 34 - DNA, 39 bp, Artificial sequence
  Synthetic construct
cgatcggatccgccaccatggaacaaaagctgatctctg

SEQ ID NO: 35 - DNA, 36 bp, Artificial sequence
  Synthetic construct
gatcatgcggccgctcaggtctggaagttggtggtc

SEQ ID NO: 36 - DNA, 40 bp, Artificial sequence
  Synthetic construct
ggaattagatctgccaccatggaacaaaagctgatctctg

SEQ ID NO: 37 - DNA, 36 bp, Artificial sequence
  Synthetic construct
aattcctctagatcacatgaacaccttatgtccgcc

SEQ ID NO: 38 - DNA, 20 bp, Artificial sequence
  Synthetic construct
gtcgccagccgagccacatc

SEQ ID NO: 39 - DNA, 20 bp, Artificial sequence
  Synthetic construct
ccaggcgcccaatacgacca

SEQ ID NO: 40 - DNA, 20 bp, Artificial sequence
  Synthetic construct
ggaggaattctcggctggag

SEQ ID NO: 41 - DNA, 19 bp, Artificial sequence
  Synthetic construct
cgttatcggggtcgacgtt

SEQ ID NO: 42 - DNA, 20 bp, Artificial sequence
  Synthetic construct
ttgcagggaacccaactacc

SEQ ID NO: 43 - DNA, 23 bp, Artificial sequence
  Synthetic construct
tccttccattgagatgaatgtgg

SEQ ID NO: 44 - DNA, 20 bp, Artificial sequence
  Synthetic construct
ttgtctcctggtcacgaacc

SEQ ID NO: 45 - DNA, 20 bp, Artificial sequence
  Synthetic construct
tgtggcagcaactcaacaga

SEQ ID NO: 46 - DNA, 21 bp, Artificial sequence
  Synthetic construct
gatgaatgtgtgagtcagacc

SEQ ID NO: 47 - DNA, 21 bp, Artificial sequence
  Synthetic construct
gcagacgatacatcaggatac

SEQ ID NO: 48 - DNA, 20 bp, Artificial sequence
  Synthetic construct
tggagactctcagggtcgaa

SEQ ID NO: 49 - DNA, 20 bp, Artificial sequence
  Synthetic construct
ggattagggcttcctcttgg

SEQ ID NO: 50 - DNA, 20 bp, Artificial sequence
  Synthetic construct
ccagggccttccttgtatct

SEQ ID NO: 51 - DNA, 18 bp, Artificial sequence
  Synthetic construct
gcatgggttctgacggac

SEQ ID NO: 52 - DNA, 20 bp, Artificial sequence
  Synthetic construct
ttcatgtacggctccaaggc

SEQ ID NO: 53 - DNA, 20 bp, Artificial sequence
  Synthetic construct
tgtagatgaactcgccgtcc

SEQ ID NO: 54 - DNA, 20 bp, Artificial sequence
  Synthetic construct
ggacgacggcaactacaaga

SEQ ID NO: 55 - DNA, 20 bp, Artificial sequence
  Synthetic construct
ttgtcgctgatggcgttgta

SEQ ID NO: 56 - DNA, 20 bp, Artificial sequence
  Synthetic construct
cttgcctggaccagcttaat

SEQ ID NO: 57 - DNA, 20 bp, Artificial sequence
  Synthetic construct
caagccgactctccatacct

SEQ ID NO: 58 - DNA, 19 bp, Artificial sequence
  Synthetic construct
acctgaaaaatggcttcgg

SEQ ID NO: 59 - DNA, 20 bp, Artificial sequence
  Synthetic construct
gtagctgcggaaaacctcct

SEQ ID NO: 60 - DNA, 20 bp, Artificial sequence
  Synthetic construct
gcaaaacaggactggctgat

SEQ ID NO: 61 - DNA, 20 bp, Artificial sequence
  Synthetic construct
actgcagaagatcccgacaa

SEQ ID NO: 62 - DNA, 20 bp, Artificial sequence
  Synthetic construct
ggcctttgacttcgttaagc

SEQ ID NO: 63 - DNA, 20 bp, Artificial sequence
  Synthetic construct
ccacctcagtgacacagcac

SEQ ID NO: 64 - DNA, 18 bp, Artificial sequence
  Synthetic construct
cacccgtgcagatgagga

SEQ ID NO: 65 - DNA, 20 bp, Artificial sequence
  Synthetic construct
ctgctggaagaggttgacca

SEQ ID NO: 66 - DNA, 20 bp, Artificial sequence
  Synthetic construct
acttcaggctcctgggcaac

SEQ ID NO: 67 - DNA, 22 bp, Artificial sequence
  Synthetic construct
cagcaagaaagcgagcttagtg

SEQ ID NO: 68 - DNA, 19 bp, Artificial sequence
  Synthetic construct
aatcgtgcgtgacattaag

SEQ ID NO: 69 - DNA, 20 bp, Artificial sequence
  Synthetic construct
atgatggagttgaaggtagt

SEQ ID NO: 70 - DNA, 21 bp, Artificial sequence
  Synthetic construct
catgctttccttcccattgtt

SEQ ID NO: 71 - DNA, 20 bp, Artificial sequence
  Synthetic construct
atgggcacgatttcttcaaa

SEQ ID NO: 72 - DNA, 20 bp, Artificial sequence
  Synthetic construct
ctggagatgggacagcaaga

SEQ ID NO: 73 - DNA, 20 bp, Artificial sequence
  Synthetic construct
atgggcacgatttcttcaaa

SEQ ID NO: 74 - DNA, 20 bp, Artificial sequence
  Synthetic construct
gggatatagacgggagccat

SEQ ID NO: 75 - DNA, 20 bp, Artificial sequence
  Synthetic construct
ccaccgatatccagtccaaa

SEQ ID NO: 76 - DNA, 23 bp, Artificial sequence
  Synthetic construct
ccctagcgtttgaaataagttgc

SEQ ID NO: 77 - DNA, 20 bp, Artificial sequence
  Synthetic construct
ccaccgatatccagtccaaa

SEQ ID NO: 78 - DNA, 19 bp, Artificial sequence
  Synthetic construct
attgtgctggatgccgaga

SEQ ID NO: 79 - DNA, 21 bp, Artificial sequence
  Synthetic construct
acctagcgtggtcactccgta

SEQ ID NO: 80 - DNA, 20 bp, Artificial sequence
  Synthetic construct
tgtctaaagccacccctctg

SEQ ID NO: 81 - DNA, 20 bp, Artificial sequence
  Synthetic construct
ctgcatggcatactcctgct

SEQ ID NO: 82 - DNA, 20 bp, Artificial sequence
  Synthetic construct
gggaaaccaaagtctttggg

SEQ ID NO: 83 - DNA, 21 bp, Artificial sequence
  Synthetic construct
ggaattaaccagacaaatcgc